(19) United States
(12) Patent Application Publication
(10) Pub. No.: US 2015/0193597 A1
(43) Pub. Date: Jul. 9, 2015

(54) PHARMACEUTICAL PRODUCT AND COMMUNICATION TOOL

(71) Applicant: SCIENTIFICMED SWEDEN AB, Stockholm (SE)

(72) Inventor: Johan Cederlund, Bromma (SE)

(73) Assignee: SCIENTIFICMED SWEDEN AB, Stocholm (SE)

(21) Appl. No.: 14/417,371

(22) PCT Filed: Jul. 12, 2013
(86) PCT No.: PCT/SE2013/050894

§ 371 Date: Jan. 26, 2015

(30) Foreign Application Priority Data
Jul. 24, 2012 (SE) 1250894-1

(51) Int. Cl. G06F 19/00; A61K 31/135; A61K 38/28; A61K 31/197; A61K 31/439; A61K 31/155; A61K 31/519

(52) U.S. Cl. CPC G06F 19/363; G06F 19/326; G06F 19/3456; G06F 19/3481; A61K 31/155; A61K 31/135; A61K 31/519; A61K 31/197; A61K 31/439; A61K 38/28

(57) ABSTRACT

A substance with pharmaceutical activity against a medical condition for use in a treatment of the medical condition in combination with a computer program product including instructions causing a computer to perform a method including:
providing a patient with a set of questions according to a question schedule, wherein the set of questions is adapted to the pharmaceutical product;
collecting answers to the questions from the patient;
subjecting the answers to a set of functions adapted for the set of questions and the pharmaceutical product thereby generating patient-specific feedback information;
providing the feedback information to the patient; and extracting information from the answers and providing the information to a database adapted to collect information during clinical use of the substance, and store a multitude of relevant information.

### FIELD OF THE INVENTION

The present invention relates to the field of drug administration, and particularly to combination products for management of drug administration and improvement of usage and clinical efficacy of pharmaceutical products in clinical practice.

### BACKGROUND

Drugs on the market today are thoroughly tested with regard to their efficacy and safety during extensive clinical trials before they are approved for marketing by a national or regional Medical Products Agency, such as EMA in Europe or FDA in the U.S.

An important aspect of the clinical trials is to achieve an optimal dosage and administration regimen and these aspects are strictly controlled and monitored during the trials. During clinical trials the manufacturers of a drug collect a large amount of data on the drug. However, once the drug is on the market the control of the dosage is in many cases left to the patient undergoing therapy. This may lead to difficulties in individualizing the used dosage of pharmaceutical products to patient specific conditions and lack of compliance to the prescribed dosing, such as under-dosage, over-dosage and gaps in the administration regimen, which leads to unsatisfactory therapeutic results of the treatment.

Drugs on the market today are stand alone products without any support or connection to the vast amount of data generated during the research and development phase of the product, which could be used for simplifying and optimizing the relation between patient needs and pharmaceutical product clinical conditions. The guidance for matching patient specific conditions to the use of pharmaceutical products is limited.

One of the major issues to reach an increased clinical effect of pharmaceutical treatments in clinical practice is to improve adherence to prescribed medication, see World Health Organisation 2003 Report: Adherence to long-term therapies; Evidence for action: http://whqlibdoc.who.int/publications/2003/9241545992.pdf Due to the lack of adherence to medication the results of pharmaceutical treatments in clinical practice have difficulties in reaching the same results in clinical effect as the ones made in clinical trials during the development of the pharmaceutical products.

In regulations from FDA and EMA focus on patient safety and follow-up of side effects, as well as possible adverse events, regarding pharmaceuticals is crucial. In clinical practice, however, this is difficult to achieve and a major responsibility is on the patient with little or no support to accomplish it properly.

Even though the safety concerns of medications are directly related to the specific pharmaceutical products, today there are very limited features, or no features, at all integrated with the pharmaceutical product aiming at improving the patient safety concerns of the product. The major responsibility for patient safety for specific pharmaceutical products is on the patients themselves.

Medical devices enhancing the therapeutic effect of drugs are known. For instance, specifically designed inhalers are used to administer various anti-asthmatic drugs and implantable devices have been used for controlled release of anti-cancer drugs.

Patient compliance and monitoring systems are known in the art, e.g. WO02095352. Such systems are focused on monitoring patient compliance and reporting to the medical practitioner and the patient how the treatment is progressing. The system disclosed in WO02095352 is relevant for a certain condition (menopause) and a general therapy (hormone replacement therapy). It is not specifically adapted for a certain pharmaceutical product.

Different types of e-health applications are existing knowledge, as well as, the positive clinical effects of such systems. This kind of applications is focused on improving the health situation for the patient in general independent of any specific pharmaceutical. This kind of system has a large interest within clinical practice, but the broad usage of such systems today within healthcare is absent.

### SUMMARY OF THE INVENTION

As stated above, large amounts of data on a pharmaceutical product are collected during clinical trials performed by the manufacturers of the pharmaceutical product. The amount of data is generally too large to be kept in the mind of a single person and is summarised by various methods into guidelines for use, such as dosage regimens, counter-indications and risks for side effects and adverse events.

A medical practitioner prescribing the pharmaceutical product, as well as a pharmacist selling a prescription or non-prescription pharmaceutical product, will have a certain knowledge of the product. In some countries lacking adequate regulations, pharmaceuticals may even be provided to patients by persons without proper pharmaceutical or medical training. The providing person's knowledge of the pharmaceutical product is based mainly on the manufacturer's information, which in turn is based on the summaries of the amount of data collected during clinical trials. The providing person may further be highly specialised in the use of the product, such as a researcher with a special interest in the product and the disease it is aimed at treating, but is more likely to be a practitioner that on a daily basis treats patients with very disparate conditions and diseases. Such a practitioner may need to stay current with information on hundreds of various pharmaceutical products. This entails that certain information, such as recently discovered information, on the product may be overlooked or unknown to the providing person. In this sense, the present invention aims to provide a technological support to the patients in order that they benefit from the most recent information about their medication, adapted to their specific situation.

The present invention is based on the realization that the integral combination of a pharmaceutical product and a specifically adapted system for receiving information from a user of the pharmaceutical product and providing feedback to said user can be used to achieve a number of benefits in clinical practice. In this way, a patient using the pharmaceutical product can directly benefit from the entire body of knowledge, such as clinical data, related to the pharmaceutical product in the possession of the manufacturer or supplier of the pharmaceutical product, in addition to the information provided by the medical practitioner and/or pharmacist providing the pharmaceutical product.

One aspect of the invention is a substance with pharmaceutical activity against a medical condition for use in a treatment of said medical condition in combination with a computer program product comprising instructions causing a computer to perform a method comprising the steps
providing a patient with a set of questions according to a question schedule, wherein said set of questions is adapted to the pharmaceutical product;
collecting answers to said questions from said patient;
subjecting said answers to a set of functions specific for the set of questions and the pharmaceutical product thereby generating patient-specific feedback information;
providing said feedback information to the patient; and
   extracting information from said answers and providing said clinically relevant information to a database adapted for collecting information during clinical use of said substance, wherein said database is adapted to store information comprising one or more of: patient identifier, respondent identifier, individual caregiver identifier, organizational caregiver identifier, substance identifier, substance combination identifier, respondent answers, type and date of occurrence of adverse events, type and degree of adverse effects of one or more substance or substance combination, probability of an adverse event, probability of an adverse effect, patient health status, patient history, patient family history, patient genetic information, prescribed dosage or administration regimen, drug-drug interactions, life-style factors.

Preferred embodiments are described in the dependent claims.

The specific information which the database is adapted to store provides the provider of the invention the possibility to collect relevant data from a significant number of patients using the invention in clinical practice and iteratively improve and further adapt the sets of questions and sets of functions to real-life conditions.

One aspect of the invention is a combination product, or a kit-of-parts, comprising the drug in question and a computer program product comprising instructions causing a computer to provide the patient with the questions, receiving answers to the questions, processing the answers and providing feedback to the patient.

One aspect of the invention is a method of treatment of a medical condition with a substance having a pharmaceutical activity against said medical condition in combination with a computer program product comprising instructions causing a computer to provide the patient with the questions, receiving answers to the questions, processing the answers and providing feedback to the patient.

The above three aspects of the invention shall be considered as equivalent unless specifically indicated otherwise. In particular, the characteristics of the pharmaceutical products and computer program products are the same in all three aspects.

Another aspect of the invention is to make clinically relevant information obtained during clinical use, i.e. clinical trials or clinical practice, of the pharmaceutical product come to the benefit of individual patients in a more efficient way. This is realized by continuously updating the Question-Feedback Model implemented in the Computer Program Product by including therein instructions causing the computer to perform a method comprising the steps
a) providing a patient and optionally a further respondent with sets of questions according to a question schedule, wherein said sets of questions are specific to the pharmaceutical product;
b) collecting answers to said questions from said patient and optionally said further respondent;
c) subjecting said answers to a set of functions specific for the sets of questions and the pharmaceutical product thereby generating patient-specific feedback information;
d) providing said feedback information to the patient and optionally to the further respondent;
e) extracting information from said answers and providing said information to said database adapted for collecting information during clinical use of said substance;
f) providing information stored in said database to a reviser subjecting the sets of questions and/or the sets of functions to a revision based on said information stored in said database;
g) obtaining a revised set of questions and/or a revised set of functions from said reviser; and
h) repeating steps a)-g).

The information on which the review is based could be collected from the individual patient or from more than one patient, preferably at least 50%, such as at least 75% or substantially 100% of patients, clinically using said substance in combination with said computer program product. Revision of the set of functions may include a revision of the feedback information and type of feedback given to the patient.

The reviser performing the revision may be one or more persons skilled in analysis of clinical data and drafting clinical guidelines, such as a team of medical doctors, clinical statisticians and/or pharmacists. It may also be a suitable computer-implemented expert system or set of revision functions. Such a set of revision functions may include comparison of patient parameters and/or patient trend lines with reference parameters and reference trend lines calculated from the information collected from more than one patient, preferably at least 50%, such as at least 75% or substantially 100% of patients, clinically using said substance(s) in combination with said computer program product. Alternatively, the reference parameters and reference trend lines are calculated from information collected only from comparable patients, e.g. patients having the same or similar age, life-style, clinical status, clinical history, sex, ethnicity etc.

One aspect of the invention is to enhance the match between the specific conditions for each particular patient, both concerning behavioural and physiological aspects, with the clinical conditions for the specific pharmaceutical product concerning used dosage, identified side effects and adverse events, and clinical effect in order to improve individualization. This may be done by including existing clinical research data for the pharmaceutical product in the combination product.

One aspect of the invention is to enhance patient compliance to the prescribed dosage or administration regimen and to enhance the clinical efficacy of the pharmaceutical product. This may be done by including questions on the actual administration; actual dosage; perceived and/or measured therapeutic effects; test results and/or perceived quality of life and providing the patient with feedback correlating the positive effects of the pharmaceutical product, and/or the absence or low prevalence of negative effects, with compliance to the prescribed dosage or administration regimen.

One aspect of the invention is to give the user early indications of the occurrence or development of a possible adverse event and/or side effect, by including questions relating to occurrence or development of a possible adverse event and/or side effect. This increased awareness of adverse events and side effects results in enhanced protection of patients from adverse events and side effects. This may enable an increased patient safety, which is demanded from authorities like EMA and FDA on pharmaceutical products. This may enable early introduction of pharmaceutical products with an incomplete safety profile on the market, since it allows for making each user of the pharmaceutical product aware of the occurrence or development of a possible adverse event and/or side effect and also facilitates that this may be reported directly to medical staff. It may also enable re-introduction of products withdrawn from the market due to an unacceptably high frequency of adverse events or side effects by making each user of the pharmaceutical product aware of the occurrence or development of a possible adverse event and/or side effect at an early stage.

One aspect of the invention is to enhance the patient's quality of life.

The computer program product is preferably adapted to be installed on a handheld device, such as a mobile telephone, a Personal Digital Assistant (PDA), tablet computer or similar devices. The computer program product may also be installed on a remote computer, e.g. a server, web or cloud-based service, and accessible to the user through a computer such as a handheld device, a stationary computer, a laptop or the like. In such a case the feedback is also preferably provided through the same device.

Other aspects of the invention are the computer program product itself and the method performed by the computer program product.

Other aspects of the invention are as provided in the appended claims.

### BRIEF DESCRIPTION OF THE DRAWINGS

FIG. 1 is a schematic overview of the combination product according to the invention.

FIG. 2 shows an alternative embodiment of the invention.

FIG. 3 shows an alternative embodiment of the invention.

FIG. 4 shows examples of the user interface of the implemented QFM in a regular mobile phone used in the three studies. Questions shown are examples of (A) Visual Analogue scale (B) multiple choice (C) numeric.

FIG. 5 shows examples of patient specific feedback. (A) Text message to patient (B) Graphs with patient specific data (C) Graphs with patient specific data, user interface on a regular computer.

FIG. 6 shows a schematic view of the development of a Question-Feedback model (QFM).

FIG. 7 shows an overview embodiment of QFM in the three first studies in the examples.

FIG. 8 shows a schematic view of Set of Questions and Feedback Information.

FIG. 9 shows a schematic view of a question schedule.

FIG. 10 shows an overview of a technical implementation of the computer program product.

FIG. 11 shows an illustrative example of one of the patient's feedback graphs from study 1 in the examples.

FIG. 12 shows an illustrative example of one of the patient's feedback graphs from study 2 in the examples.

FIG. 13 shows an illustrative example of one of the patient's feedback graphs from study 3 in the examples

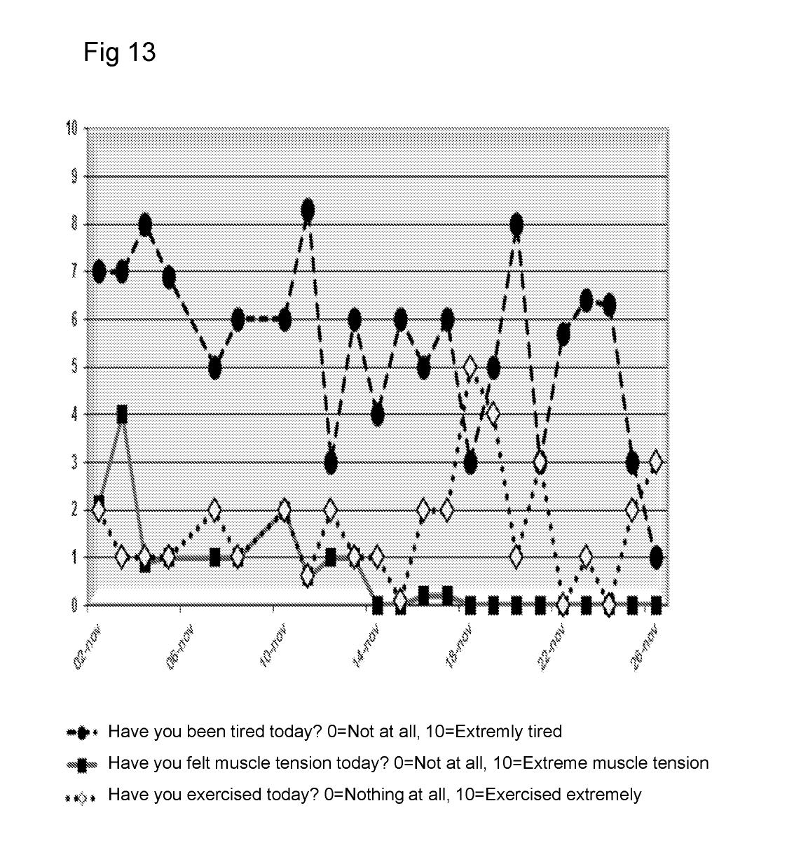

### DEFINITIONS

All words and terms used in the present specification are intended to have the meaning usually given to them in the relevant art. However, for the sake of clarity, a few terms are specifically defined below.

The term “set of questions” is a questionnaire with predetermined questions or items shown to a respondent to get answers for feedback purposes. The questions within the set preferably have a limited number of possible answers, such as yes/no; scale 1-10; multiple choice; etc. The questions may however also have an undefined number of answers, such as a value of a test parameter (e.g. blood pressure, blood glucose level).

The questions in the set of question are posed to the respondent according to a certain regimen or schedule. This is denoted a “question schedule” or “question regimen” in the present application. These terms are intended to be equivalent if not otherwise indicated.

The term “set of functions” means a set of functions that can be applied to the answers to a set of questions to extract specified information and generate feedback based on the answers.

The combination of a set of questions and a set of functions is referred to as a “question-feedback model”, sometimes abbreviated “QFM”.

That a set of questions is “specific” to a certain pharmaceutical product shall be construed to mean that it comprises questions that are applicable and clinically relevant to the pharmaceutical product. The individual questions, and the set of questions in total, are preferably more applicable and clinically relevant to the pharmaceutical product in question than to any other pharmaceutical product.

The term “respondent” is used to denote the individual responding to a question.

The term “patient” is used to denote the individual using the pharmaceutical product.

The terms “pharmaceutical product” and “medical product” shall be considered equivalent unless specifically indicated otherwise. These terms refer to pharmaceutically acceptable compositions of pharmaceutically active substances (drugs) intended for administration to a patient.

The term “side effect” means a secondary and usually adverse effect of a drug or treatment.

The term “adverse event” means an adverse outcome that occurs during or after the use of a drug or other intervention but is not necessarily caused by it.

“Clinical use” shall be construed as the use of the pharmaceutical product by individual subjects. It includes the use of the pharmaceutical product in Phase I, II and III clinical trials and the use of the product in patients in clinical practice (sometimes referred to as Phase IV clinical trial).

“Clinically relevant information” shall be construed as information relevant to the clinical characteristics of a pharmaceutical product, e.g. on effect, side effects, counter-indications, metabolism etc. Such information is extensively collected during clinical trials.

### DETAILED DESCRIPTION OF THE INVENTION

The main aspect of the present invention is a combination product comprising a pharmaceutical product and a computer program product comprising instructions to perform a method comprising the steps of providing a defined set of specific questions to the user, collecting answers to the questions and analyzing, transforming and processing the answers by way of a defined set of specific functions to generate feedback to the patient.

By adapting the combination of the set of questions and the set of functions, which combination is hereinafter called the “question-feedback model”, to be specific to the pharmaceutical product, and optionally the therapeutic indication and/or prescribed dosage/administration regimen, it is possible to achieve an unexpected and significant improvement in the therapeutic effect of the pharmaceutical product and quality of life for patients. Without being bound by theory, the improved therapeutic effect of the pharmaceutical product and quality of life may be due to improved individualization concerning patient specific conditions and clinical aspects of the pharmaceutical product, due to improved compliance by the patient to the prescribed administration and/or dosage regimen, due to improved awareness of other factors influencing the relevant condition being treated with the pharmaceutical product, or due to a placebo or placebo-like effect.

For each combination of the computer program product and the pharmaceutical product a question-feedback model is developed and adapted to the specific characteristics of the pharmaceutical product and the behavior of the patients within the actual therapeutic area(s). The development of the question-feedback model follows the same general rules for different types of pharmaceutical products, but the specific question-feedback models will be different due to the characteristic of the pharmaceutical product and its clinically relevant information.

The question-feedback model comprises the following parts:

A set of questions specific for the pharmaceutical product. The set of questions is implemented in a questionnaire giving the respondent the ability to choose any of a number of possible answers to each question or enter a number representing a test value. The questions may relate to the following, the list being illustrative and non-exhaustive:
Side effects and adverse events, such as adverse drug effects
Compliance to dosage and/or administration regimen, such as if or when the pharmaceutical product has been administered; or what dose was administered.
Symptoms, such as stiffness; swelling of limbs or joints; fatigue; dizziness; headache; pain; blood in excrement; incontinence; fever; urticaria; rashes; skin irritation; itching; anxiety; dryness of mouth or other mucosa; shortness of breath; coughing; sneezing; rhinitis; irritation; restlessness
Food and drink consumption, such as meal size; meal frequency; type of diet; satisfaction with diet
Exercise, such as type, duration, frequency or avoidance of physical exercise
Mood, such as if the respondent is happy, sad, depressed, anxious, restless, etc.
Sleep, such as if the patient has slept well; duration or quality of sleep
Use of tobacco, alcohol and other drugs, such as type and amount of use; urge to use; intention or inclination to quit use; progress or lack of progress in cessation
Stress, such as perceived stress level; amount of personal quality time or spare time; amount of family quality time
Body functions, such as function of the gastrointestinal system; mental capacity, muscle strength/weakness; olfactory capacity; cardiovascular capacity
Treatment, such as if the treatment is perceived as working well; motivation to start or continue treatment
Quantitative test results, such as blood pressure; body fluid or excrement analysis results; body weight; Body Mass Index; pulse
General, such as quality of life; feeling of support from family, friends, caregiver

The questions within the set preferably have a limited number of possible answers, such as yes/no; Visual Analogue Scale (VAS); Likert scale; multiple choice, including symbols (such as “happy face” and “sad face” to capture mood); etc. The questions may however also have an undefined number of answers, such as a value of a test parameter (e.g. blood pressure, blood glucose level, body temperature, weight) or free text.

Generally, the questions are posed to the patient using the pharmaceutical product because only the patient has the true first-hand knowledge of his or her situation. However, in addition to questions posed to the patient, further questions may be posed to other respondents. These may include family members, relatives or other persons close to the patient. This may be particularly useful for pharmaceutical products used in treatment of psychiatric disorders where the patient's assessment of his or her situation may be incomplete and observations made by another person may be valuable. Questions to be answered by other respondents may belong to the same set of questions as those answered by the patient, but may be implemented in a separate questionnaire.

The specific questions and invitations given to the respondents and the type of questions are adapted to the specific characteristics of the pharmaceutical product and the behavior of the patients within the therapeutic area in order to optimize the clinical effects.

When defining the actual questionnaire it is preferable to develop questions to the respondent in order to identify possible upcoming adverse events, or indications of adverse events, as well as possible upcoming side effects with the purpose of increasing patient safety of the specific pharmaceutical product.

In addition to the set of questions, also a regimen for asking the respondent questions should be developed, including which questions are compulsory to answer, optionally before or after a certain time or within a certain time interval; which questions may be left unanswered; at what time of day the questions will show up for the respondents to answer them; with what frequency the questions shall show up etc. The regimen can be static over time but also change, e.g. the frequency of questions can decrease with time or change depending on the respondent's answers.

In addition to the above described questions it may be advantageous to include messages, which cannot be answered, to the respondent. Such messages may include recommendations, suggestions or information intended to motivate the respondent, e.g. to continue the prescribed dosage regimen although symptoms have disappeared or are less pronounced.

It may furthermore be advantageous to adapt the set of questions and messages and the regimen for asking the questions and providing the messages with regard to cultural differences and the language of the user. Principles for the translation and cultural adaptation process for PRO measures have been described (Wild D, et al., Value Health 2005; 2:94-104) and may be adapted to the present invention by the skilled person.

The question-feedback model further comprises retrieving answers from the respondents in a predefined format suitable for input into the set of functions for generating feedback.

The question-feedback model further comprises a set of functions to generate patient-specific feedback based on the answers of the respondent or respondents. These functions may comprise:
Calculations resulting in a realistic target for a specific patient to achieve. The target could be based on information given from the results from earlier clinical trials concerning the pharmaceutical product. The target could then, for example, be illustrated as a continuous graph of the predicted development for the patient, given that the prescribed administration or dosage regimen is followed. The illustration of this continuous graph would vary between different pharmaceutical products and therapeutic areas. In some areas it would illustrate the improvement of the condition whereas in other areas, for example, COPD (Chronic Obstructive Pulmonary Disease) where patients slowly degenerates, it would illustrate the lack or relative slowness of degeneration.
Calculations of future predictions for a specific pharmaceutical product and patient, based upon earlier answers from the patient and results from clinical trials and answers from other patients in real life using the actual pharmaceutical product, for example external web and data sources. These future predictions could, for example, be several predictions for each patient, based upon different circumstances in the shape of how the patient changes his/her behavior. An example of this could be if the patient increase the adherence/compliance to the specific pharmaceutical product, the patient will develop in a more positive way concerning specific symptoms of the disease.
Knowledge and rules using, for example, methods for Computer Adaptive Testing and Item Response Theory including adapted databank with the purpose of optimal individualized and personalized medicine. This could, for example, result in an individualized questionnaire for each patient based upon their own characteristics and behavior.
Calculation of trend lines based upon the specific pharmaceutical product and the answers given by the patient.
Rules and thresholds for defining when to give notifications concerning the pharmaceutical product and different kind of issues, e.g. possible adverse events, possible side effects, change dosage regimen, possible interaction of other prescribed drugs etc. These have to be carefully developed and take notice of possible combination between different questions, the evolvement of the answers from patients over time, other possibly used medication, etc.

Patient-specific feed-back is generated by the above described set of functions based on answers supplied by the patient. The feedback may be provided through any medium favorable to the patient, e.g. through a website, a handheld device (mobile phone, tablet computer, PDA, etc), paper, voice, e-mail, telefax, SMS, or corresponding type of message etc.

Examples of feedback are:
Graphs illustrating the answers given by the patient on different selected questions. The graphs may, among other things, illustrate how the patient has evolved over time.
Illustrating the answers from the patient in combination with calculated values such as targets for the patient to reach. The purpose of this type of feedback is, for instance, to motivate the patient to continuous improvements.
Illustrations of how the patient's health status is evolving in comparison to the evolvement of earlier patients using the same pharmaceutical product, for example patients in clinical trials.
Illustrations of how the patient's health status could evolve and the result of it as future prediction, based upon how the patient continues to handle his/her health situation and data from clinical use of the pharmaceutical product. For example, graphs could be used to show how the patient might evolve if the patient increased the adherence/compliance to the medication of the pharmaceutical product.
The, preferably de-identified, answers from the patient in relation to calculations based upon information given from other patients in real life/clinical practice using the pharmaceutical product, specifically selected for the actual circumstance. The purpose of this is, among other things, to encourage the patient to increase the personal health status.
Message sent based upon notifications from the algorithms. This could, for example, be messages concerning possible adverse events, or indications of possible side effects, or possible conclusions that a new dosage for the actual pharmaceutical product might be needed, or positive feedback to the patient to encourage a behavior leading to e.g. better compliance or increased quality of life. Exemplary messages could include messages that the used dosage of the pharmaceutical product should be changed, or that the alcohol consumption is below or above a recommended threshold for the pharmaceutical product, or that the amount of consumed food is high or low in comparison to physical activity, or that the first signs of a side effect appear to be showing and that the patient should be aware of these signs. The invention will hence enable a faster change of used medicines by patients experiencing an adverse event. The patient can receive messages from the healthcare personnel as well through the computer program product, as a result of the feedback given to them.
The questionnaire given to the patient could change based upon the algorithms for CAT and IRT (see above), or other appropriate algorithms or computer implemented methods, in order to individualize the questions for the characteristics of each patient and the pharmaceutical product.

Optionally, feedback may also be provided to other than the patient, such as health care staff (e.g. treating medical practitioner or nurse, pharmacist etc.). Such feedback may include:
Results from notifications from the algorithms, e.g. when an adverse event or a side effect has occurred. This information could, for example, be sent to the responsible healthcare provider and/or authorities such as the Medical Product Agency. The healthcare personnel will then be able to take appropriate adjustments. The graphs and illustrations presented above could be given to the responsible healthcare personnel as well.
Results from continuous results in real clinical trials based upon the answers given by the patients. The invention could hence improve clinical research through continuous follow up of a huge amount of patients for the specific selected pharmaceutical products. The information/answers from the patients would be de-identified and returned to the researching organization. The purpose is to utilize the enormous information in real clinical practice in order to develop improved pharmaceutical products and treatments for patients.

The continuous follow-up of the results from patients will also result in possibilities for an easy evaluation between different kind of treatments, both from a medical and an economic perspective.

The question-feedback model may be adapted to the specific pharmaceutical product by using the information on the pharmaceutical product available from clinical trials carried out in preparation for an application for marketing approval for the pharmaceutical product. Such trials are designed to find all relevant information about the pharmaceutical product and that information can be used to design the set of questions with applicable answers, the set of functions for generating the feedback from the answers, and the form of feedback provided to the patient. The continuous development of the QFM, for a specific pharmaceutical product, should also take into consideration relevant knowledge from clinical practice concerning the specific pharmaceutical product, other studies, patient behavior concerning the specific pharmaceutical product, etc.

Information on the normal effect of the pharmaceutical product can be used to provide the patient with feedback on how he or she achieves a better or worse effect than normal when using the pharmaceutical product. It may also be used to give the patient feedback on how the treated condition would have developed if the pharmaceutical product had not been used, or used to a different extent than the patient is actually using it.

Information on known possible side effects may be used to include questions giving early feedback on occurrence of side effects, which may guide the user to change or cease the administration or dosage regimen according to guidelines based on the information about side effects, or to contact the treating physician if advised.

Information on known counter-indications for using the pharmaceutical product may be used to include questions giving early feedback warning for possible side effects or adverse events. It may be that during treatment with the pharmaceutical product the patient contracts a condition which may lead to an adverse event or side effect in combination with the pharmaceutical product. If such risks are known, it is possible to include questions resulting in feedback making the patient and optionally the treating physician aware of this complication, which may lead to an adjustment or change in treatment implying an improved patient safety of the pharmaceutical product.

For example, one specific pharmaceutical product indicated for treatment of obesity is known to worsen depressions. The majority of questions and feedback in a question-feedback model for an obesity drug would probably focus on diet, physical activity, weight loss and the like. The inclusion of one or more mood-related questions would however be able to indicate early if the patient is at risk of developing a depression which would be a strong indication to the patient to cease the administration of the pharmaceutical product. These questions should be specifically designed to retrieve relevant information on the types of mood-related adverse events or side effects associated with the specific pharmaceutical product.

Optionally, additional information not supplied directly by the patient is used. This may include
Information from performed clinical trials. This could, for example, be the result how the included patients in the clinical trials using the actual pharmaceutical product responded to the pharmaceutical.
Information from other patients in clinical practice. This could, for example, be the result and answers given by other patients in real life using the equivalent pharmaceutical product and how they respond to the pharmaceutical. Using that information, a common index of how a huge amount of patients react upon the actual pharmaceutical product in real life can be evaluated, for instance.
Information from other products and systems, such as administration systems, laboratory data, personal patient devices such as watches, heart rate monitors, scales, mobile phone applications, pedometers, glucose meters, thermometers, audiometers, inhalers, ultrasound devices, electrocardiography devices, etc. Such information can be automatically collected by or transferred to the computer program product by different means.

For each combination of a specific pharmaceutical product and the computer program product a candidate specific question-feedback model has to be developed. This candidate model has to be developed based on all considerations mentioned above.

The development of the candidate question-feedback model includes the following steps:

An optimal set of questions is identified and developed. The intention should be to develop an optimal set of questions and normally this is an iterative process. In this, the following aspects should be considered, as well as the concerns mentioned above describing what is included in the set of questions.
The set of questions should be designed based upon the specific circumstances of the pharmaceutical product concerning the existence of possible adverse events, possible side effects and the therapeutic effect.
The set of questions should be designed based upon the special circumstances of the patient category of the actual therapeutic area.
The set of questions should be designed in order to improve the behavioral aspects of the patients. They should increase the possibilities for enhanced clinical effect and patient safety of the specific pharmaceutical product, and the quality of life for the patients.
The questions should be easy to understand and encourage the patient to answer them. The suitable and optimal structure type of questions should be used, i.e. VAS, Likert scale, free text, multiple choice, etc.
The amount of questions should be minimized in order to simplify for the patients.
The proper regimen for asking the respondent questions should be developed. The following should, for example, be defined:
   When the questions should appear in the patient's device, for instance which specific day and what time during the day
   Which questions that should be compulsory to answer
   The frequency of how often the questions should appear in the patient's device
Which questions that should be able to individualize, i.e. to add or remove, and to which extent. For example, some questions could be able to appear more or less seldom, i.e. changing the frequency of the question. Possibilities to support life style changes of the patients, central to the specific pharmaceutical product, e.g. within the metabolic syndrome for cardiovascular pharmaceutical products.
Whether, and in which way, the set of questions should be individualized and adopted based upon patient and pharmaceutical product specific conditions. This could involve how the questions should be answered, selection of media, etc, with the purpose of improving the clinical effect and patient safety of the specific pharmaceutical product.

An optimal set of functions is identified and developed. The intention should be to develop an optimal set of functions and normally this is an iterative process. In this, the following aspects should be considered, as well as the concerns mentioned above describing what is included in the set of functions.
The set of functions should be designed based upon the specific circumstances of the pharmaceutical product concerning the existence of possible adverse events, possible side effects and the therapeutic effect.
The set of functions should be designed based upon the special circumstances of the patient category of the actual therapeutic area.
The set of functions should be designed in order to improve the behavioral aspects of the patients. They should increase the possibilities for enhanced clinical effect and patient safety of the specific pharmaceutical product, and the quality of life for the patients.
The set of functions should be developed based upon which type of information that is possible to use considering the specific pharmaceutical product, e.g. if there are information from earlier clinical trials and/or if information from other patients in clinical practice, that can be utilized.
The set of functions should be developed based upon whether knowledge and rules from methods using Item Response Theory and Computer Adaptive Testing, or other appropriate algorithms or computer implemented methods, are available.
The set of functions concerning rules and thresholds, for example with the purpose of avoiding possible adverse events and/or side effects, giving positive feedback and optimizing the dosage regimen, should be developed concerning the circumstances of the pharmaceutical product, performed clinical trials and the specific patient population.
The set of functions could contain rules of which questions should be related to specific thresholds, for example if a threshold is reached by a patient, which questions should then appear or which type of feedback should be given
The set of functions could contain dependencies between certain questions and the functionality and rules of the dependencies, e.g. if a patient answers a specific alternative on one question another specific question appear, otherwise another question will appear instead.
The set of functions could contain the administration rules concerning different intervals when specific questions will appear based on a certain threshold, which could be time or that a criterion has been fulfilled. An example of this is that during a first period of time the patient could have a certain set of questions, and after a certain time, which could be a couple of weeks or months, the set of questions changes into another version. The set of questions could also be changed due to a certain threshold has been fulfilled, for example a certain level of blood pressure or the level of HbA1c is reached.

An optimal type of feedback should be identified and developed. The intention should be to develop an optimal type of feedback and normally this is an iterative process. In this, the following aspects should be considered, as well as the concerns mentioned above describing what is included in the type of feedback.
The type of feedback should be designed based upon the specific circumstances of the pharmaceutical product concerning the existence of possible adverse events, possible side effects, and the therapeutic effect.
The type of feedback should be designed based upon the special circumstances of the patient category of the actual therapeutic area.
The type of feedback should be designed in order to improve the behavioral aspects of the patients. They should increase the possibilities for enhanced clinical effect and patient safety of the specific pharmaceutical product, and the quality of life for the patients.
It should be defined which type of feedback that should be given to whom.
The type of feedback should be designed and developed based upon whom to which the feedback should be given to.
The type of feedback should be designed and developed based upon the developed set of questions and set of functions for the specific question-feedback model.
The type of feedback could be designed to improve the clinical effect and patient safety of the specific pharmaceutical product in using the given thresholds
The type of feedback could be designed in order to improve the clinical effect and patient safety of the specific pharmaceutical product by individualizing the dosage administration of the specific pharmaceutical product to the conditions of the patient

It may be desirable to furthermore optimize the set of questions and the feedback for use on a certain computer platform. For instance, if the respondent will use a simple mobile telephone the questions will be adapted so that they can be answered simply by pressing buttons 0-9 and yes/no/up/down and feedback may be provided in short text messages and simple graphs. If the respondent uses an advanced mobile telephone or tablet computer the questions may be constructed to give more complex answers and still be easy to use, and the feedback may also be made more complex, such as color-coded graphs and longer messages.

The candidate question-feedback model is then validated in one or more steps. The validation of the model aims to evaluate and ensure the therapeutic effect of the integrated combination of the computer program product and pharmaceutical product, minimize the amount of adverse events and side effects, and increase the quality of life for the patients. The evaluation of the clinical efficacy and value of the candidate question-feedback model for a specific pharmaceutical product is preferably performed through clinical trials, in what is usually referred to as a Phase II clinical trial or a corresponding study. In this the candidate question-feedback model for the pharmaceutical product is evaluated concerning clinical efficacy such as positive medical efficacy and increased security level for the combination product.

There are a number of types and designs of clinical trials and a skilled person would be able to choose a type of trial and design well suited to achieve the aims as outlined herein. The clinical trials or corresponding study should be designed to focus to prove the following of the model enabling the combination of the computer program product and the pharmaceutical product:
achieve optimum medical efficacy of the combined product
achieve optimum level of safety for patients
increase quality of life for the patient

Based on progress and results from clinical trials and clinical practice, the question-feedback model may of course be adjusted or revised in order to improve its efficacy, safety or other aspects of quality.

The combination of the question-feedback model and the pharmaceutical product may also be compared to an existing approved treatment in a Phase III-type clinical trial before being put on the market.

The question-feedback model is implemented in one or more computer-program products running on one or more computer platforms, wherein the computer program product and the computer platform together have means for providing the set of questions, for receiving the answers, for applying the set of functions to generate the patient-specific feedback and preferably also for providing said feedback to the patient.

The computer program product may be supplied on a suitable carrier together with the pharmaceutical product, as a kit-of-parts. Suitable carriers are well-known to the skilled person and depend on the platform on which the computer program product shall run, but includes without limitation, CD-ROM, USB-memory sticks, flash memory cards. The computer program product may also be made available to the end user separately from the physical pharmaceutical product. This can be done e.g. by supplying information on how to access the computer program product on a remote server and install the computer program product on the relevant platform with the pharmaceutical product. The computer program product could also run on a remote server and be accessed via an internet service using a user interface like a web browser or client application for the relevant platform. Ways of accessing and implementing the computer program product could also include barcode scanning techniques. The computer program product may be included in the kit-of-parts in the form of instructions for accessing and/or installing the computer program product from a remote location, such as a remote server. Information about how to get started with the computer program product and how to use it could be given in the instructions related to the pharmaceutical product or the computer program product.

If the computer program product is made available separately from the pharmaceutical product, a unique identifier may be provided with each individual kit. The identifier may be used to confirm that the respondent has got the correct combination of computer program product and pharmaceutical product and to confirm that the respondent has the right to use the computer program product.

The computer program product is an essential part of the main aspect of the invention and is itself one aspect of the invention, as is the method implemented in the computer program product.

The pharmaceutical product may be any pharmaceutical product for which there exists a preferred or prescribed administration and/or dosage regimen. This includes all pharmaceutical products that have been approved for marketing based on results of clinical trials defining a therapeutically effective dose or dose range and pharmaceutical products for which a medical or other practitioner prescribes an individual administration or dosage regimen to an individual patient based on information supplied by the manufacturer of the pharmaceutical product. It furthermore includes pharmaceutical products for which an application for marketing approval is to be submitted, pending, or has been refused. The pharmaceutical product may or may not be subject to regulation by a Medical Products Agency or other governmental agency, it may be a prescription only product, an over-the-counter product or any other allegedly therapeutically active product, such as a herbal medicinal product.

Examples of pharmaceutical products that can be used in the present invention are (trade names within parentheses) Aripiprazol (Abilify)Rimonabant (Acomplia), Pioglitazon (Actos), glucoseamine (Glucosine), Octocog alfa (Advate, Advair), Flutikason in combination with Salmeterol (Seretide), zolpidem (Ambien, Stilnox), Insulin glulisin (Apidra), Donepezil (Aricept), irbesartan (Avapro, Aprovel), rosiglitazone (Avandia), metformin in combination with rosiglitazone (Avandamet), glimepiride in combination with rosiglitazone (Avandaryl), bevacizumab (Avastin), Interferon beta (Avonex), Darbepoetin alfa (Aranesp), anastrozole (Arimidex), Kandesartan (Atacand), olmesartan (Benicar, Olmetec), Interferon beta-lb (Betaseron), Interferon beta (Betaferon), exenatide (Byetta), Bikalutamid (Casodex), Celecoxib (Celebrex, Celebra), Escitalopram (Cipralex/Lexapro), duloxetine (Cymbalta), Vareniklin (Champix), Glatiramer (Copaxone), Carvedilol (Coreg), Losartan (Cozaar), Rosuvastatin (Crestor), Ramipril (Tritace), Valsartan (Diovan), Venlafaxin (Efexor), oxaliplatin (Eloxatin), Etanercept (Enbrel), raloxifene (Evista), ezetimibe (Ezetrol, Zetia), Tamsulosin (Flomax, Flomaxtra, Urimax), fluticasone (Flovent, Flixotide), Alendronic acid (Fosamax), Gemcitabine (Gemzar), imatinib mesylate (Gleevec, Glivec), Trastuzumab (Herceptin), insulin lispro (Humalog), Adalimumab (Humira), Lopinavir/ritonavir (Kaletra), Sumatriptan (Imitrex, Imigran), Sitagliptin (Januvia), insulin glargin (Lantus), Fenofibrate (Lipanthyl, TriCor), atorvastatin (Lipitor), Insulin Detemir (Levemir), amlodipine and benazepril (Lotrel), Leuprorelin, (Lupron, Leuplin), pregabalin (Lyrica), rituximab (Mabthera, Rituxan), Telmisartan (Micardis), Esomeprazole (Nexium), amlodipine (Norvasc), insulin aspart (NovoLog, NovoMix, NovoRapid), repaglinid (NovoNorm), Rabeprazole (Pariet), paroxetine (Paxil, Seroxat), Pantoprazole (Protonix, Pantozol, Pantoloc), Clopidogrel (Plavix), pravastatin (Pravachol), Epoetin Alfa (Procrit, Eprex), takrolimus (Protopic), budesonid (Pulmicort), interferon beta-la (Rebif), sibutramin (Reductil), Infliximab (Remicade), Risperidon (Risperdal), Metoprolol (Seloken, Toprol), quetiapine (Seroquel), Tiotropium (Spiriva), budesonide and formoterol (Symbicort), Montelukast (Singulair), Docetaxel (Taxotere), Topiramat (Topamax), Emtricitabin and Tenofovirdisoproxil (Truvada), ezetimibe and simvastatin (Vytorin), bupropion (Wellbutrin), Betametason in combination with Kalcipotriol (Xamiol) calcipotriene (Taclonex), simvastatin (Zocor), Sertralin (Zoloft), zoledronic acid (Zometa), Olanzapin (Zyprexa), cetirizine (Zyrtec), ticagrelor (Brilique).

The invention will now be described in relation to the appended drawings.

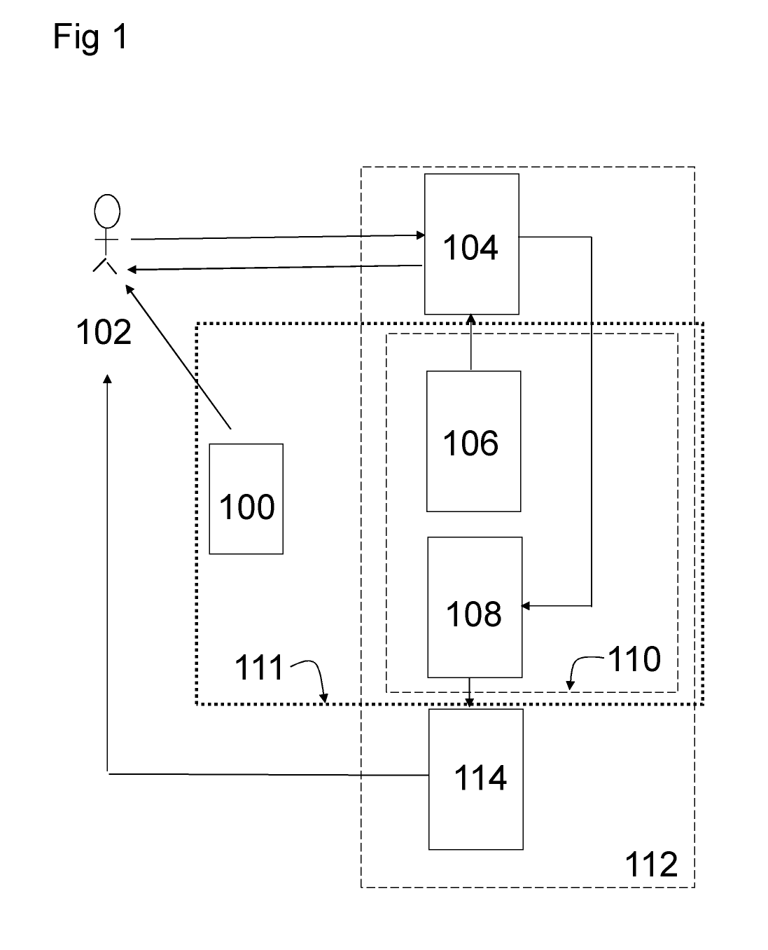

FIG. 1 shows a combination product (111) comprising a pharmaceutical product (100) available to a patient/respondent (102) and a computer program product (110). A set of questions (106) and a set of functions (108) for converting the answers to the questions into patient feedback are implemented in the computer program product (110) running on a computer platform (112) having means (104) for receiving answers to said set of questions (106) from said patient (102). The computer platform further has means (114) for receiving patient feedback from the set of functions (108) and communicating said feedback to said patient (102). The combination product according to the invention is designated as 111

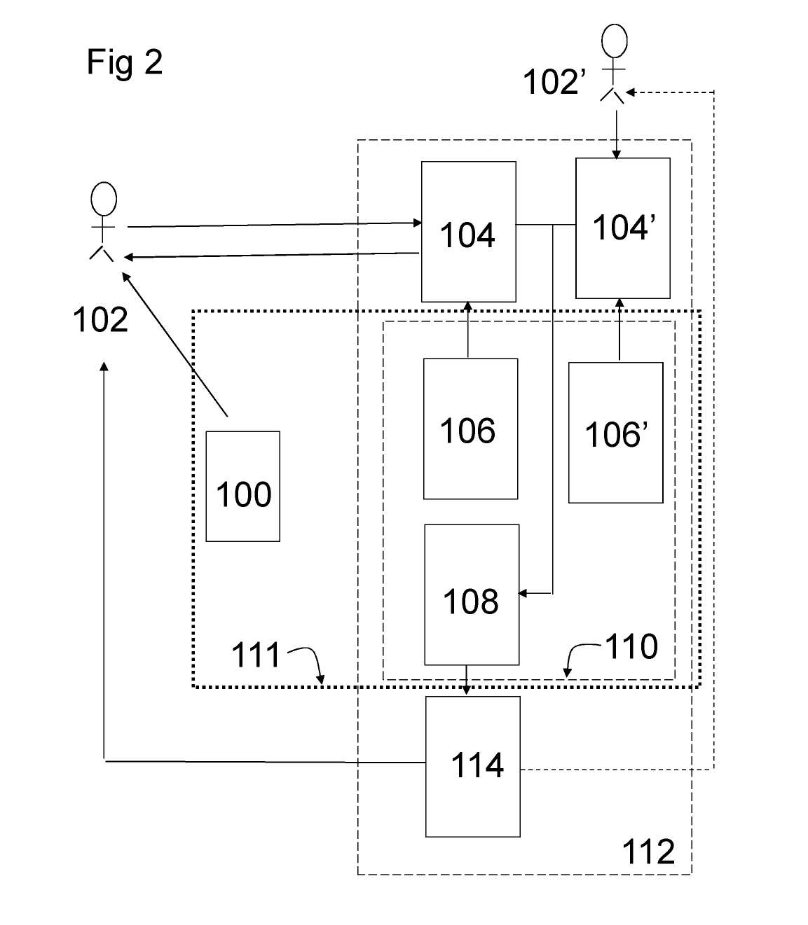

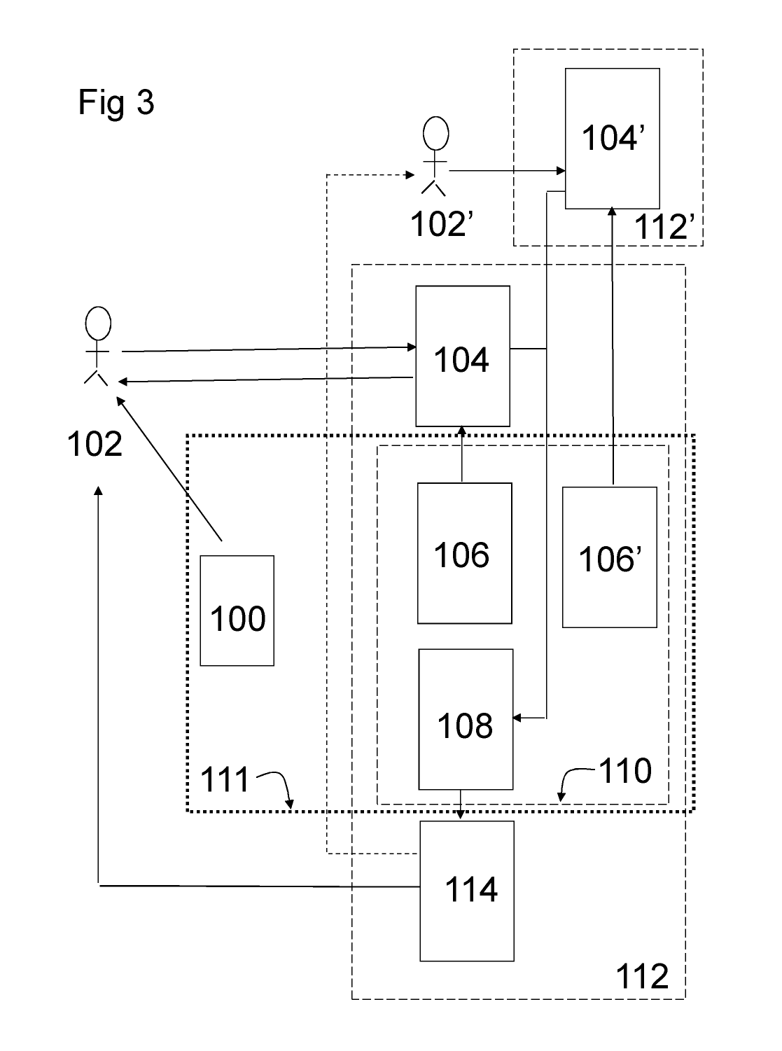

FIG. 2 shows an alternative embodiment of the invention, wherein a further respondent (102′) answers a second set of questions (106′) through means (104′) for receiving answers to said set of questions from said further respondent. The answers to the set (106′) is then provided together with the answers to the set (106) to the set of functions (108) to generate feedback to patient (102) through computer platform means (114) for receiving patient feedback from the set of functions (108) and communicating said feedback to said patient (102). Optionally, feedback is also provided to the further respondent (102′), shown with a dotted line. The further respondent may be a person close to the patient, such as a family member. The means (104′) for receiving answers from the further respondent may be implemented on a separate computer platform (112′), cf FIG. 3.

The examples below serve to further illustrate the invention, provide experimental support and enable the skilled person to work the invention. They shall not be construed as limiting the scope of the invention, which is that defined by the appended claims.

### EXAMPLES

The implementation of the invention in clinical practice is described below in four examples relating to various pharmaceutical products aimed at treating various medical conditions. The examples are provided in order to give a further explanation of the invention but are not intended to limit the scope of the invention, which is that of the appended claims.

Common Descriptions for the Three Studies

We have performed three studies to show how the invention works and the positive effects of the invention. Studies 1-3 describe the use of an initial QFM that is adapted to the pharmaceutical product but not yet fully optimized. This shows that the invention works and gives a tangible clinical effect. Further optimization of the QFM will yield a better clinical effect.

In the studies the combination product, a computer program product (CPP) integrated with a pharmaceutical product (PP) using an adapted question-feedback model (QFM), were evaluated versus only a PP, respectively versus only a CPP. The overall purpose was to evaluate different aspects of the invention in three different therapeutic areas, and using three different types of a PP, in order to show the effect of the invention.

In order to visualize the study designs, objectives and results as clearly as possible; in the studies the used PP is denoted as the letter “A”, the used CPP as the letter “B”, and the combination product, i.e., a specific PP in combination with a CPP using an adapted QFM, as the letters “A+B”.

General Objectives of the Studies

Several important aspects of the invention have been evaluated in the three separate studies in different therapy areas; diabetes, atopic dermatitis, and generalized anxiety disorder (GAD). In table 1 below the different evaluations in the three studies are summarized.

TABLE 1
Summary of the evaluations of the invention

| Evaluation | Therapy area | Effect variable A + B |
|---|---|---|
| Clinical effect of A + B versus only A | Diabetes | Level of HbA1c |
| Clinical effect of A + B versus B | Atopic dermatitis | Primary symptom and side effects |
| Perceived clinical value of A + B over | Atopic dermatitis | Perceived clinical value of A + B |
| time | | |
| Clinical effect of A + B versus A | GAD | Primary symptoms |
| concerning improved clinical value | | |
| Clinical effect of A + B versus A | GAD | Primary symptoms and side |
| concerning improved patient safety | | effects |
| Quality of life of A + B versus only A | GAD | Perceived quality of life |
| Adherence to A when using A + B | GAD | Level of adherence to A |
| compared to only A | | |

Conclusions and General Aspects Concerning the Results from the Studies

The results from the studies confirm that the invention works and that the combination product, A+B, gives the following positive effects:
1. Improved clinical effect
2. Improved patient safety
3. Increased quality of life

For detailed description concerning the specific results, see the study documentation below. One central aspect was the improved efficacy when identifying and realizing an individualized dosage regimen for the given PP. In the GAD study this was clearly illustrated, using the combination product. When the dosage of the used PP was individualized, based on the used QFM, not only was the clinical effect improved but also the patient safety. In this particular case the decisions were to either increase the dosage of the PP or interrupt the usage of it.

Another aspect of the increased clinical effects mentioned above was the increased level of compliance to the prescribed PP, which the use of the combination product led to. This was also illustrated in two different perspectives in the studies of atopic dermatitis and GAD. The former showed an increased perceived level of practical usage and the latter showed an increased adherence.

The improved clinical result, especially when it came to diabetes, was also due to an improved awareness of other factors relevant to the actual therapy area, the patient population and the specific PP. Such factors included levels of physical activity, stress, and food intake.

Another aspect of the invention and the results from particularly the GAD study was the central role of the QFM. The QFM had to be specific both to the conditions of the patient category and to the clinical effect of the PP, in order to achieve a better clinical effect than just from the PP alone. In the GAD study it was obvious that the set of functions and feedback were a central part of the invention in order to achieve clinical effect. An example of the opposite situation was the result from one of the Atopic study set-ups, when a patient was using just “B” without an adapted QFM. Another aspect of the invention is the mechanism of improved patient safety regarding side effects, adverse events, and dosage regimen of the specific PP. A key mechanism is to continuously measure, detect, and follow up clinical effect, side effects, and adverse events in clinical practice. Another key mechanism is the increased awareness the measuring (questions and feedback) routine gives the patient about his/her health situation and medical treatment concerning central aspects of the specific PP. Among other things it helps the patient to understand and detect possible side effects and adverse events. The mechanisms form a basis for well-based decision-making for a possible titration, interruption, or other reaction of the medication treatment.

Another aspect of the studies, and in particular the atopic dermatitis study, was the patients' desire for even more feedback regarding the use of the PP.

Another aspect of the three studies was that the invention created improved positive clinical effect concerning PPs in three totally different therapy areas, which shows the great width of the invention. Particularly it is possible to improve clinical effect both in therapy areas where the measurement variables (e.g. symptoms and side effects) are relatively concrete and absolute, such as the situation with diabetes, and in therapy areas where the measurement variables are relatively subjectively, such as the situation with GAD.

Another aspect of the invention and the results is that it is valid for different types of PP. In the three studies the PP had different pharmaceutical compositions; a capsule, an ointment, and an injection.

General Aspects of the Used QFM

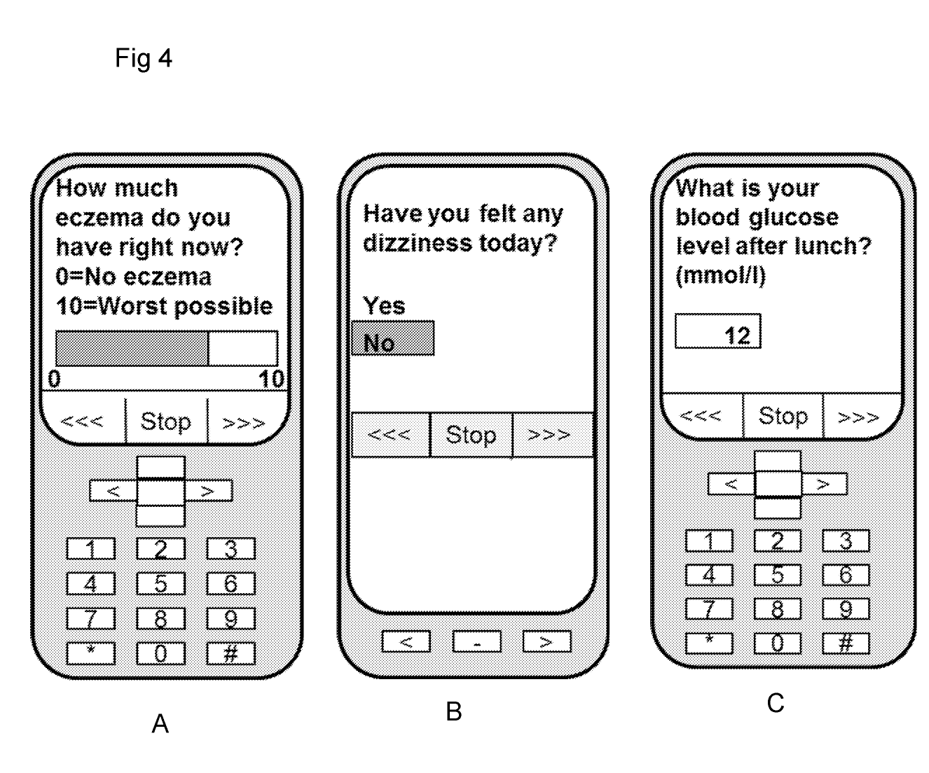

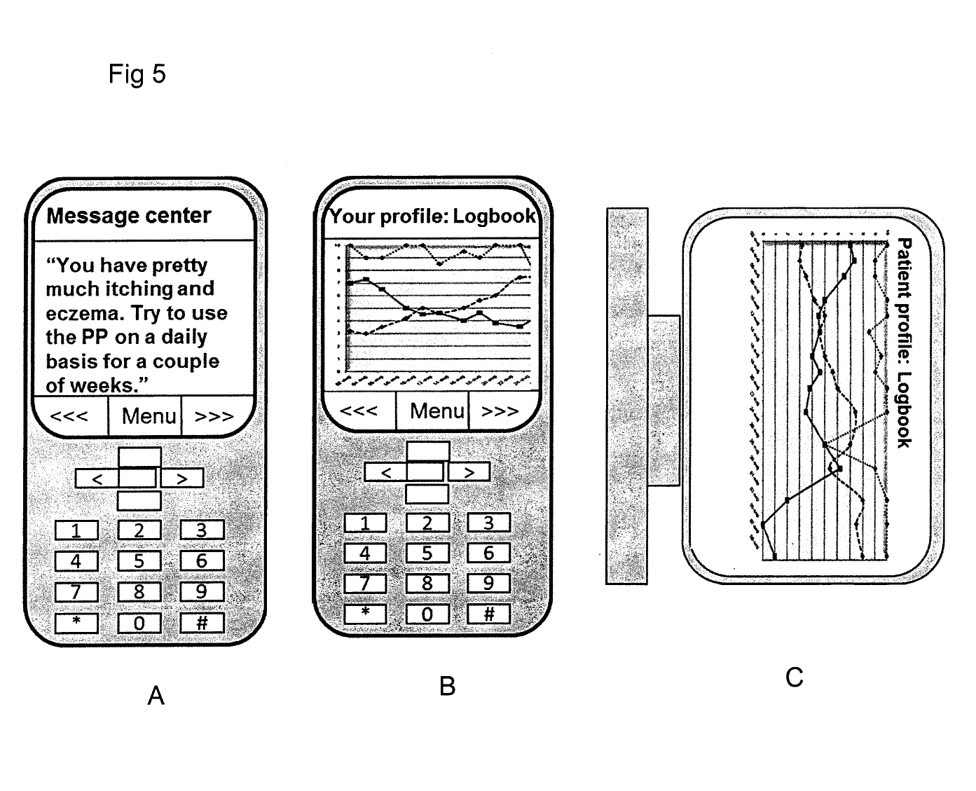

In the three studies the respectively used QFM consisted of the following parts:
A set of questions. Some of the characteristics:
   Developed based on the specific aspects of the PP and the patient category.
   One compulsory group of questions to be asked, which was given to all patients, and one optional group of questions to be asked if they were relevant for the individual patient.
   The questions could be individualized depending on the patients' specific conditions and situation. For example, specific questions could be added or removed depending on specific patient conditions.
   Different type of questions, i.e. multiple choice, VAS, etc.
   Both compulsory and optional to answer questions.
   The questions were integrated with a question schedule with response times. The response times included automatic reminders (alerts) in the CPP on the mobile phones to remind the patients to answer the questions. The question schedule was developed so only the questions valid for each response time showed up in the CPP and were possible for the patient to answer. This feature secured that the patients answered the right questions at the right time. The question schedule could be individualized depending on the patient's daily schedule.
   The questions were presented to the patient on the patient's mobile phone. The illustration (FIG. 4) shows examples of the user interface of the implemented QFM in a regular mobile phone used in the three studies. Questions shown are examples of Visual Analogue scale (FIG. 4A), multiple choice (FIG. 4B) and numeric (FIG. 4C).
A set of functions. Some of the characteristics:
   Calculations on the data, consisted of the answers from the patients, in order to present patient specific information in different graphs. Data from different questions were grouped together to visualize important relationships and correlations between variables. Graphs were constructed to show development over time for chosen variables.
   Calculations on the collected and non-collected data, which could trigger SMS reminders to the patients about continuously answering the questions.
   Algorithms enabling the question schedules.
   Applications handling and securing that patient specific information could only be viewed by authorized personnel.
   Applications handling and securing that feedback were realized in different digital channels such as Internet and SMS.
Patient-specific feedback information (see FIG. 5). Some of the characteristics:
   Developed based on the specific aspects of the PP and the patient category.
   Patient specific graphs based upon the collected answers from the patients to the set of questions. This type of feedback was given to the patients in the studies concerning diabetes and atopic dermatitis, not in GAD. In all three studies health care personnel had access to these patient specific graphs, which they used for giving feedback in different ways to their patients.
   The graphs were constructed in a way where relevant variables were matched together and plotted over time according to the set of functions. This showed interesting and valuable relationships and correlations that gave both the patients and/or the healthcare personnel a better understanding of the patients' situation and development.
   Patient specific SMS sent to the patients regarding their treatment and situation (all studies).
   Patient specific SMS sent to the patients with reminders to continue answering questions when their adherence to answer the questions decreased or stopped (all studies).
   Oral communication between health care personnel and the patients based on the patient specific feedback information generated by the CPP (all studies).

Development of the Used QFM

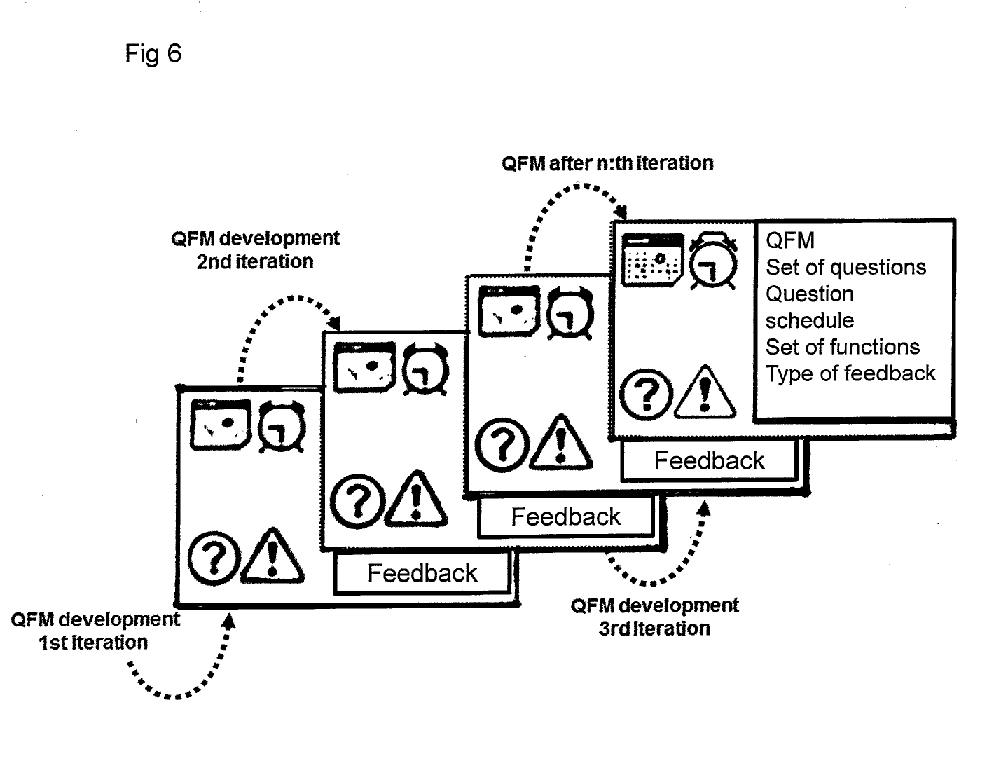

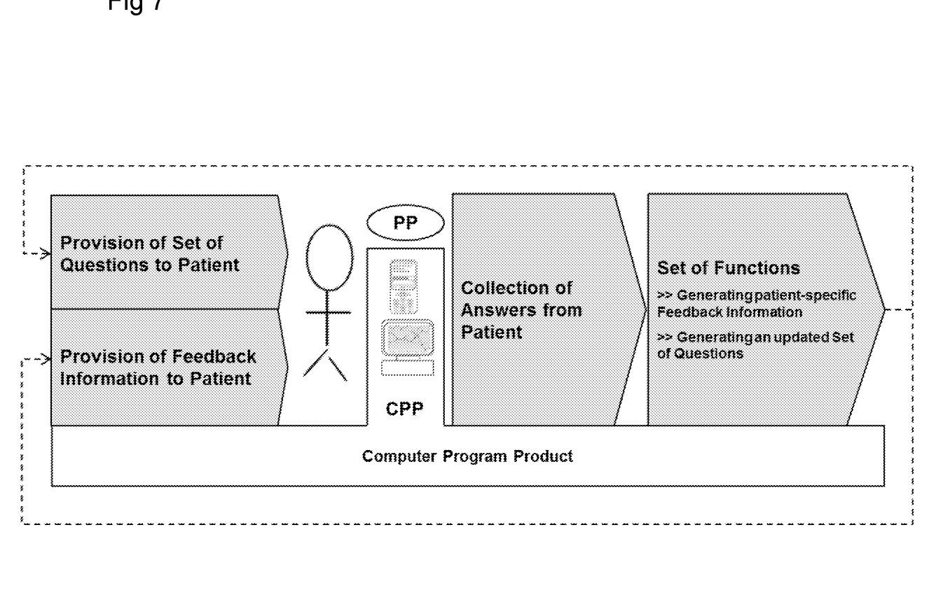

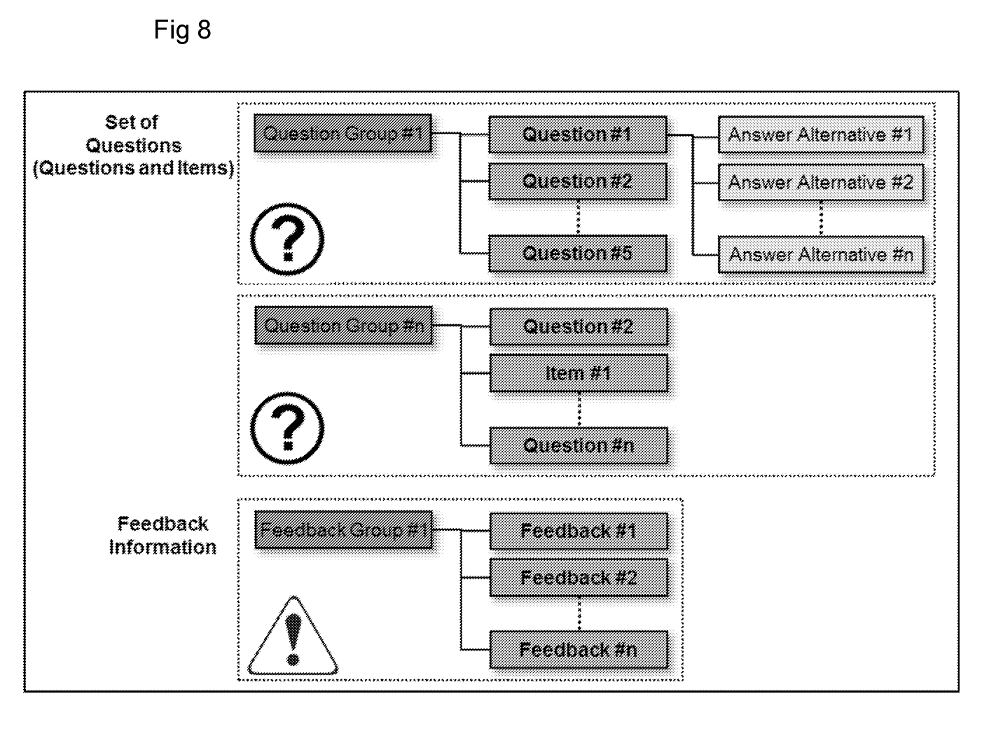

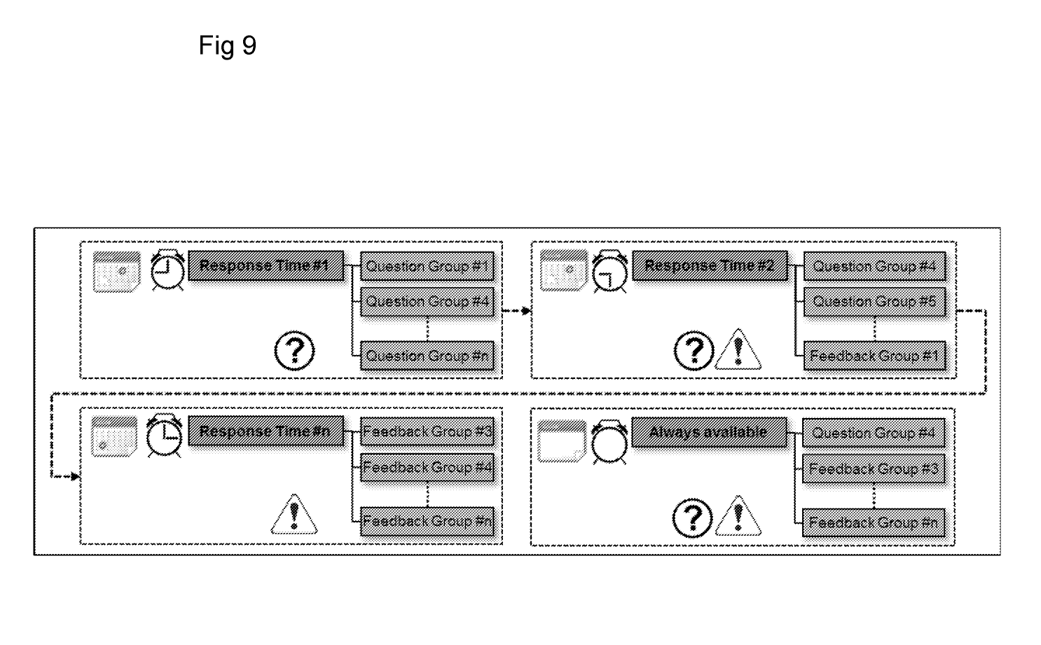

The development of the used QFM for each of the three pharmaceutical products in the three studies included mainly the steps described earlier in the detailed description and clinically relevant information of the specific pharmaceutical products. Normally it is an iterative process (see FIG. 6) before an optimal QFM for the specific PP (see FIG. 7) has been developed with the set of questions and feedback information (see FIG. 8) and the question schedule (see FIG. 9). As said earlier in the detailed description, many aspects and considerations need to be taken into account when developing a specific QFM.

Overview Technical Implementation of the CPP

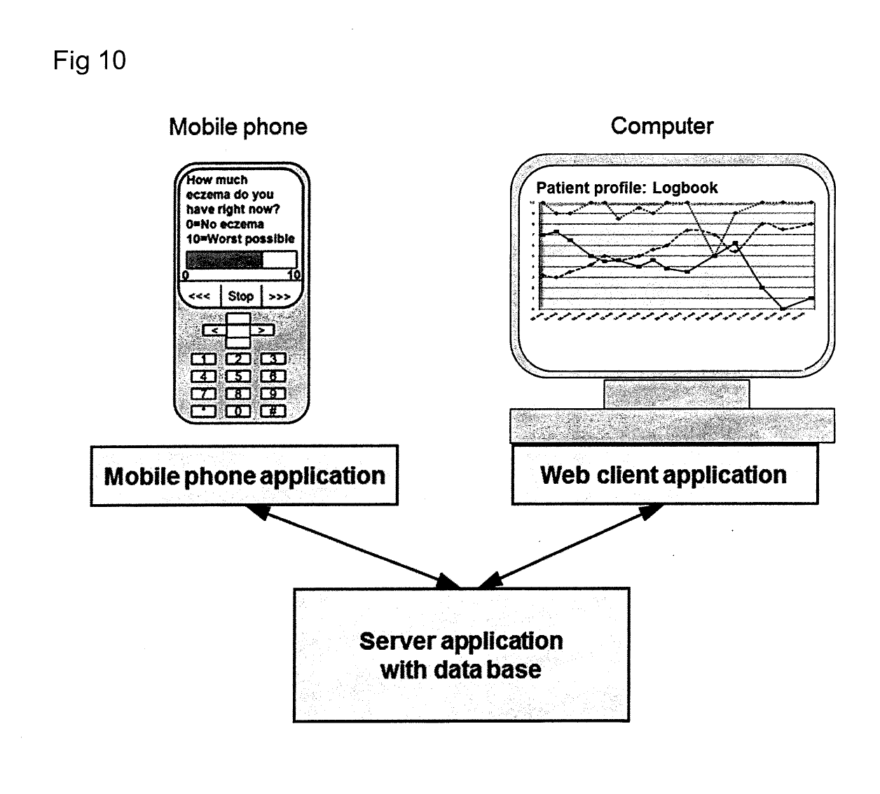

The technical realization and implementation of the CPP in the three studies is illustrated in FIG. 10. The patients were first registered in the system by the health care personnel and after that the patients could download, via mobile internet, the mobile phone application to their mobile phones. The mobile phone application could process, handle and present the questions and answers to the patient. The CPP also consisted of a web client application which was the primary user interface for the health care personnel. A server application with a data base was also an integral part of the implementation of the CPP.

Study 1. Rapid-Acting Insulin and Type 1 Diabetes

Background

Type 1 diabetes is an auto-immune disease in which the body's immune system destroys the insulin-producing beta cells in the pancreas. This type of diabetes, also known as juvenile-onset or insulin-dependent diabetes, accounts for 10-15% of all people with the disease. People with type 1 diabetes must inject themselves with insulin several times a day and follow a careful diet and exercise plan.

Glycated hemoglobin (hemoglobin A1c, HbA1c, A1C) is a form of hemoglobin that is measured primarily to identify the average plasma glucose concentration over prolonged periods of time. This serves as a marker for average blood glucose levels over the previous months prior to the measurement.

HbA1c is recommended by WHO (World Health Organization) as a test to diagnose diabetes. The American Diabetes Association recommends that the HbA1c should be below 53 mmol/mol (7.0%) for most patients.

Rapid-acting insulin begins working very quickly inside the body—usually within 5 and 10 minutes. This type of insulin should be taken just before or just after eating. It operates at maximum strength for one to two hours and duration is typically up to four hours. Rapid-acting insulin's are very convenient because they allow diabetic patients to inject themselves, at the time, when they eat.

Study Objectives

The study objective was to evaluate the clinical effect of using the combination product in type1 diabetes in comparison of using only a PP. The measured variable was HbA1c. The variable was measured directly before the patients entered into the study and directly afterwards when they had concluded their participation.

Primary variable: HbA1c.

Study Design and Set-Up

Two patients were given the combination product, A+B. Both patients had during a longer period of time (more than 6 months) prior to the study been given the specific PP, i.e. only “A”, without any significant improvement in the levels of HbA1c.

Length of study: 3 months

Number of patients: 2

Inclusion criteria: Diagnosed diabetes type1 with more than 58 mmol/mol HbA1c. Access to a mobile phone capable of handling the used CPP.

Study set up: A+B versus A. Two patients used A+B. Evaluation of change in HbA1c, before and after the study.

Used PP: Rapid-acting insulin

The used set of questions can be seen in table 2. The different questions were grouped together in questions groups with corresponding response times (see table 3). Some of the questions were asked three times a week, some more seldom, and some were “spontaneous”, i.e., always available for the patient to answer. The question regime, appeared to the patient, could be another than the one presented in the table.

TABLE 2
Questions

| | Question type and answer |
|---|---|
| Question | alternatives |
| “Have you been irritated at someone/something | VAS 0-10 |
| today?” | 0 = Not at all irritated, 10 = Extremely |
| | irritated |
| “How focused are you at school/work?” | VAS 0-10 |
| | 0 = Not at all focused, 10 = Very |
| | focused |
| “How did you sleep last night?” | VAS 0-10 |
| | 0 = Very poorly, 10 = Very well |
| “For how long time have you exercised today?” | Multiple choice: 0 min, 1-20 min, |
| | 21-40 min, 41-60 min, More than 60 min |
| “How many blood glucose levels have you checked | Numeric |
| today?” | |
| “How many units of rapid-acting insulin did you | Numeric |
| take at breakfast?” | |
| “How many units of rapid-acting insulin did you | Numeric |
| take at the meal?” | |
| “When did you eat breakfast?” | Multiple choice: Before 6 am, |
| | Between 6-8 am, Between 8-10 am, I |
| | didn't eat breakfast |
| “When did you eat lunch?” | Multiple choice: Before 11 am, |
| | Between 11 am-1 pm, Between 1-3 pm, |
| | I didn't eat lunch |
| “What was your blood glucose level approximately | Numeric |
| 1.5 hours after breakfast (mmol/l)?” | |
| “What was your blood glucose level approximately | Numeric |
| 1.5 hours after lunch (mmol/l)?” | |
| “What was your blood glucose level before | Numeric |
| breakfast (mmol/l)?” | |
| “What was your blood glucose level before lunch | Numeric |
| (mmol/l)?” | |
| “How hard is it to have been diagnosed with type 1 | VAS 0-10 |
| diabetes?” | 0 = Not at all difficult, 10 = Extremely |
| | hard |
| “To what extent has diabetes affected your | VAS 0-10 |
| activities during the week?” | 0 = Very much, 10 = Not at all |

TABLE 3
Question schedule

| Question group | Response time (alerts from CPP) |
|---|---|
| “Morning questions” | Mondays, Wednesdays, and Fridays |
| | at 10 am |
| “Afternoon questions” | Mondays, Wednesdays, and Fridays |
| | at 3 pm |
| “Weekly questions” | Once a week on Fridays at 3 pm |
| “Monthly questions” | Once a month on Fridays at 3 pm |
| “Spontaneous questions” | Questions always available to answer |

Type of Feedback

The feedback to the patients was crucial in order to achieve a positive clinical effect of the combination product.

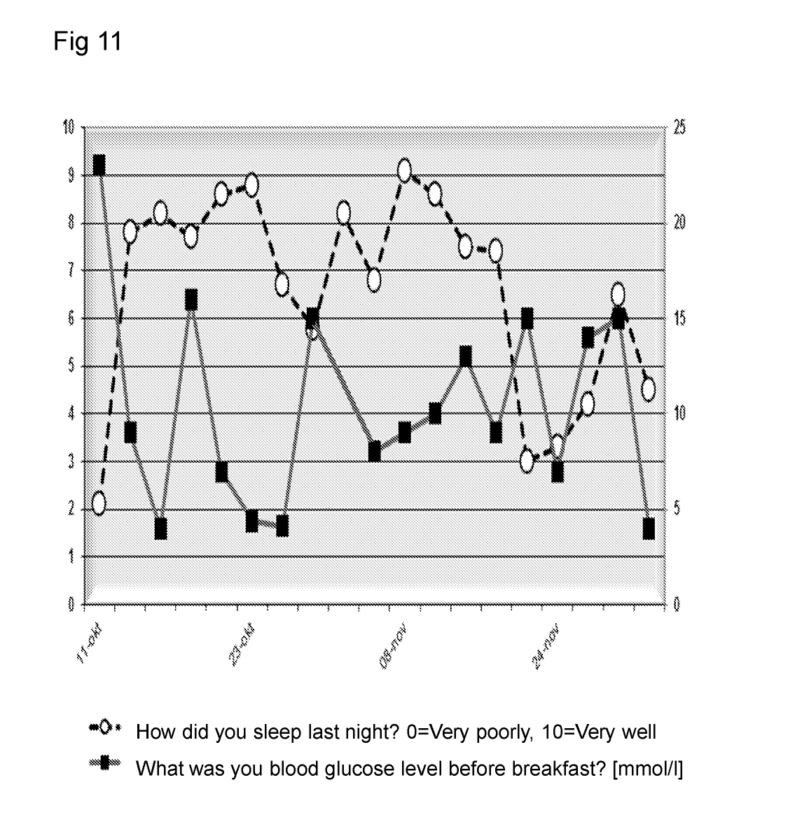

Both the healthcare personnel and the patients had access to updated graphs with the patient's specific feedback information based on the collected answers. The graphs were constructed in a way where relevant variables were matched together and plotted over time, examples of the matched variables are shown in table 4. An illustrative example with one of the patient's feedback graphs is shown in FIG. 11. Examples of given feedbacks to patients were the following text messages (SMS) sent via the CP, see table 5.

TABLE 4
Examples of grouping of variables in feedback graphs

| Grouping | |
|---|---|
| of feedback | |
| graphs | Questions/Variables |
| Blood glucose and | “What was your blood glucose level |
| insulin at breakfast | before breakfast (mmol/l)?” |
| | “How many units of rapid-acting insulin did |
| | you take at breakfast?” |
| | “What was your blood glucose level approximately |
| | 1.5 hours after breakfast (mmol/l)?” |
| Sleep and blood | “How did you sleep last night?” |
| glucose | “What was your blood glucose level |
| | before breakfast (mmol/l)?” |

TABLE 5
Illustrative examples of feedback messages

| | Number of | | Examples of SMS |
|---|---|---|---|
| | SMS- | Examples of SMS-messages from | message generated |
| Patient | messages | health care personnel | from the CPP |
| Patient 1 | 10 from health | “Hi, it would be interesting and | “Don't forget to |
| | care personnel 3 | valuable to see more test values. | answer the questions |
| | automatically | Please record more blood glucose | continuously. If you |
| | generated from | readings after breakfast and lunch.” | haven't received |
| | the CPP | “Hi, enjoy spring break, but don't | alerts from your |
| | | forget to check your blood glucose | mobile phone |
| | | levels” | recently, please try |
| Patient 2 | 9 from health | “Hi, don't forget to record your blood | to restart the |
| | care personnel 2 | glucose values 1.5 hours after | application again.” |
| | automatically | breakfast and lunch. These readings | |
| | generated from | are important.” | |
| | the CPP | “Good work! But I miss some blood | |
| | | glucose readings after your meals” | |

Study Results

The result of the study is presented in the table 6 below.

TABLE 6
Study results

| | HbA1c before | HbA1c after | |
|---|---|---|---|
| Patient | enrollment | completion | Change in HbA1c |
| Patient 1 | 106 mmol/mol | 89 mmol/mol | −17 mmol/mol |
| Patient 2 | 80 mmol/mol | 63 mmol/mol | −17 mmol/mol |

The result shows a substantial improvement in the clinical effect of the combination product, A+B, in comparison to only A. The value of the primary variable HbA1c improved significantly, 19% as an average, when the patients had been using the combination product, A+B compared to before the study when they were using only A during at least 6 months. The period of using only A for the patients resulted in the level of HbA1c measured before enrollment into the study.

The result of the study indicates a significant clinical effect of the invention, the combination product.

Study 2. Takrolimus and Atopic Dermatitis

Background

Atopic dermatitis is an inflammatory, chronically relapsing, non-contagious and pruritic skin disorder. Although there is no cure for atopic eczema, and its cause is not well understood, it can be treated very effectively in the short term through a combination of prevention (learning what triggers the skin reactions) and drug therapy.

Protopic Ointment (active substance takrolimus) is a prescription ointment used to treat moderate to severe eczema. Protopic is for use after other prescription medicines have not worked or when a doctor recommends that other prescription medicines should not be used. Protopic should be used for short periods, and, if needed, treatment may be repeated with breaks in between.

Study Objectives

The study objectives were twofold:
1. Evaluate the possible improvements in clinical effect of using the combination product, A+B compared to B.
   Measured symptoms: Perceived level of eczema and perceived level of itching.
2. Evaluate the possible perceived clinical effect in the value of the combination product.
   Measured variable: Perceived level of practical value of the combination product.

Study Design and Set-Up

Four patients were given the combination product, A+B, and one patient the CPP, just B. Prior to entering into the study none of the patients had used either the PP or the CPP.

Length of study: 3 months.

Number of patients: 5 in total. Four in the intervention group with A+B, and one in the control group with B.

Inclusion criteria: Diagnosed atopic dermatitis and access to a cellular phone capable of handling the used CPP.

Used PP: Protopic

The one patient in the control group used a cortiscosteroid based regimen instead of Protopic. The patient used the same CPP and QFM as the other patients, but this QFM was adapted to Protopic and not the cortiscosteroid based pharmaceutical product.

Two different study set-ups:
1. Comparison of A+B versus B. Four patients using A+B. Control group with one patient using just B.
2. Evaluation of A+B over time, i.e., start period compared with end period. Four patients using A+B.

The used set of questions can be seen in table 7. The different questions were grouped together in question groups with corresponding response times (see table 8). The questions were asked twice a week and they were also “spontaneous”, i.e., always available for the patient to answer.

TABLE 7
Questions

| | Question type and answer |
|---|---|
| Question | alternatives |
| “How much eczema do you have right now?” | VAS 0-10 |
| | 0 = No eczema, 10 = Worst possible |
| “How much itching did you have the last day and | VAS 0-10 |
| night?” | 0 = Nothing at all, 10 = Very severe |
| “How does the treatment practically work out for | VAS 0-10 |
| you?” | 0 = Very bad, 10 = Very good |
| “If you have eczema - how often do you anoint | Multiple choice: Daily, Twice a |
| yourself with Protopic?” | week, Not at all |
| “If you don't have eczema - how often do you | Multiple choice: Daily, Twice a |
| anoint yourself with Protopic?” | week, Not at all |

TABLE 8
Question schedule

| Question group | Response time (alerts from CPP) |
|---|---|
| “Regular questions” | Mondays and Thursdays at 7 pm |
| “Spontaneous questions” | Questions always available to answer |

The type of feedback was, as stated earlier, access to own patient specific graphs, received personal and patient specific SMS, and feedback from the health care personnel via oral communication.

Measured Variables

The measured variables (see table 9) are symptom levels which are perceived estimates by each patient at every measure point. The levels of symptoms at the beginning of the study are compared to the levels of the symptoms at the end of the study. In parallel with the study, all patients were answering a continuous follow-up question regarding the perceived value of practical functioning of using the combination product, i.e. the medical treatment combined with the computerized program.

TABLE 9
Measured variables

| Symptom/Variable | Question in QFM | Question type |
|---|---|---|
| Eczema | “How much eczema do you have right | VAS 0-10 |
| | now?” | 0 = No eczema, 10 = Worst |
| | | possible |
| Itching | “How much itching did you have the last | VAS 0-10 |
| | day and night?” | 0 = Nothing at all, 10 = Very |
| | | severe |
| Practical functioning | “How does the treatment practically work | VAS 0-10 |
| of treatment | out for you?” | 0 = Very bad, 10 = Very good |
| Measured variable | Definition | Data type |
| Eczema (average) | Average for symptom during the period | 0-10 |
| | | 0 = No eczema, 10 = Worst |
| | | possible |
| Itching (average) | Average for symptom during the period | 0-10 |
| | | 0 = Nothing at all, 10 = Very |
| | | severe |
| Practical functioning | Average for variable during the period | 0-10 |
| of treatment (average) | | 0 = Very bad, 10 = Very good |

Type of Feedback

The feedback to the patients was crucial in order to achieve a positive clinical effect of the combination product.

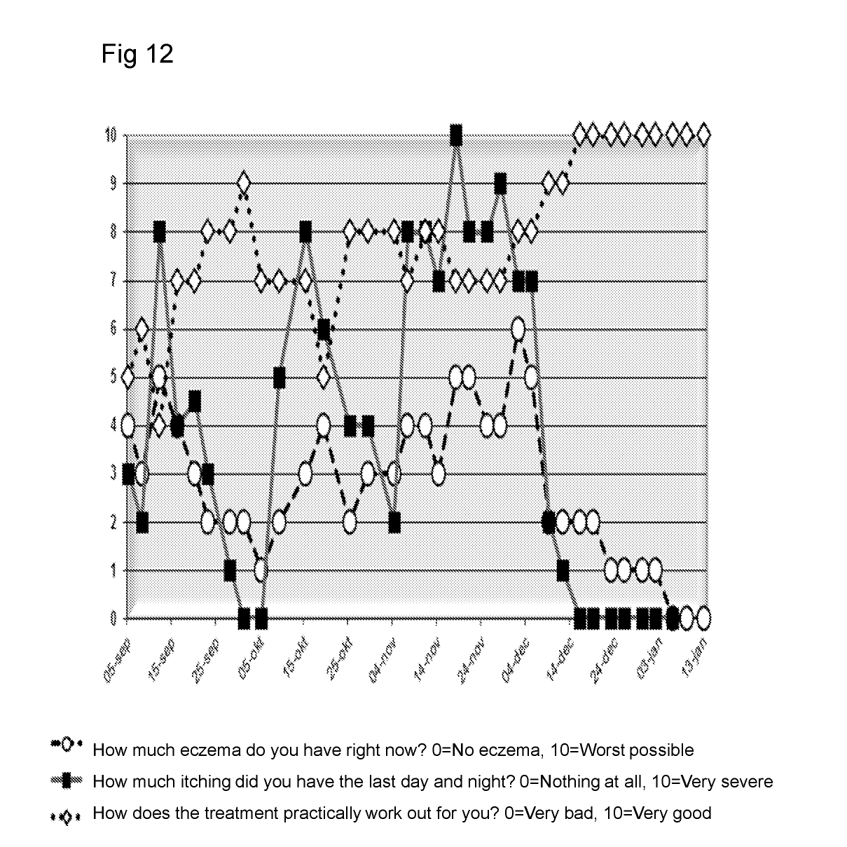

Both the healthcare personnel and the patients had access to updated graphs with the patient's own specific feedback information based on the collected answers. The graphs were constructed in a way where relevant variables were matched together and plotted over time, examples of the matched variables are shown in table 10. An illustrative example with one of the patient's feedback graphs is shown in FIG. 12. Examples of given feedbacks to patients, and subsequent actions and clinical effects, are the following text messages (SMS) sent via the CPP, to two of the patients can be seen in table 11.

TABLE 10
Example of grouping of variables in feedback graphs

| Grouping of | |
|---|---|
| feedback | |
| graphs | Questions/Variables |
| Eczema, itching, | “How much eczema do you have right now?” |
| and practical | “How much itching did you have the last day |
| treatment | and night?” |
| | “How does the treatment practically work out |
| | for you?” |
| Eczema and | “How much eczema do you have right now?” |
| adherence | “If you have eczema - how often do you anoint |
| Protopic | yourself with Protopic?” |
| | “If you don't have eczema - how often do you anoint |
| | yourself with Protopic?” |

TABLE 11
Illustrative examples of feedback messages

| | | Result of reaction to |
|---|---|---|
| Feedback | Patient reaction to feedback | feedback |
| “You have pretty much | The patient started using the PP | The measured variables itching |
| itching and eczema. Try to | on a daily basis at once. | and eczema started to improve. |
| instead use the PP on a daily | | |
| basis for a couple of weeks.” | | |
| “Your eczema and itching | From that date the patient was | The measured variables |
| seem to be in decent control. | fully adherent to the PP. | remained on a stable and very |
| If you have any question | | positive level. |
| please get in contact.” | | |

Study Results

The study results, measured variables and changes, are presented in the table 12 below. Decimal rounding has been made to the data in the table. N/A=Not applicable.

TABLE 12
Study results

| | | Eczema | Eczema | | Itching | Itching | | Practical | Practical | Practical |
|---|---|---|---|---|---|---|---|---|---|---|
| | | Start | End | Eczema | Start | End | Itching | treatment | treatment | treatment |
| Patient | Type | period | period | Change | period | period | Change | Start period | End period | Change |
| Patient 1 | A + B | 2.7 | 2.5 | −0.2 | 2.1 | 2.1 | 0.0 | 9.3 | 10 | 0.7 |
| Patient 2 | A + B | 2.8 | 0.2 | −2.6 | 3.3 | 0.0 | −3.3 | 6.6 | 10 | 3.4 |
| Patient 3 | A + B | 5.3 | 4.3 | −1.0 | 5.6 | 5.0 | −0.6 | 4.3 | 5.3 | 1.0 |
| Patient 4 | A + B | 6.4 | 5.1 | −1.2 | 6.6 | 6.0 | −0.6 | 6.4 | 7.9 | 1.6 |
| Patient 5 | B | 2.3 | 4.6 | 2.2 | 2.8 | 4.9 | 2.0 | N/A | N/A | N/A |

The result shows a significant improvement in the clinical effect of the combination product, A+B compared to B, see table 12. The level of clinical effect, concerning both the perceived levels of eczema and itching, is improving substantially; see the columns Eczema Change and Itching Change in the table above. Both measured symptoms are significantly decreasing, both in comparison with the initial values and with the progress of the control group.

The result shows also a significant improvement in perceived value of practical functionality of using the combination product over time.

Aspects Comparing A+B Versus B

The patient in the control group shows a negative result in both perceived level of eczema and itching. This result is an effect of the QFM being adapted for the specific PP. The actual patient in the control group is not using the specific PP, implying a situation where the actual QFM not being optimal for the specific patient. In order to achieve an effect of the invention, the QFM has to be adapted to the specific PP and to the specific situation for the actual patient. None of this is the case for the patient in the control group in this study.

Aspects Evaluating A+B Over Time

The patient's adherence to the whole treatment, i.e. the combination product A+B, is measured by asking how the patient perceives the practical functioning of the treatment. A major reason for that is that the usage of the PP, from a patient perspective, is done through a cumbersome procedure

From an invention perspective, the result that the perceived value is increasing over time is positive due to the following aspects:
The value of the CPP should, according to the insights behind the invention, increase over time, because it takes some time for a patient to get the full value of the QFM and the invention.
The measured variable is related to a perceived quality of life of the patient, implying that such a factor might also develop positively.
The adherence and sense of practical functioning using only a PP is normally long-term constant or decreasing.

After the study the patients asked for even more frequent feedback. They felt a value in receiving feedback about their situation and how to act in order to improve their health situation. They also wanted the set of questions even more adapted to their own specific situation as a group of patients and to the specific PP. They also wanted to have furthermore individualized questionnaires. This kind of comments supports the idea behind the invention regarding the importance of personalized feedback, and also shows that the development of the QFM is crucial in order to optimize the clinical effect.

Study 3. Pregabalin and Generalized Anxiety Disorder (GAD)

Background* *Source: European Medicines Agency, Summary of the European Public Assessment Report (EPAR) for Lyrica.

Generalized anxiety disorder (GAD) is an anxiety disorder. The symptoms of GAD are prolonged excessive anxiety and worry that are difficult to control. GAD can also cause restlessness or feeling keyed up or on edge, being easily fatigued (tired), having difficulty concentrating or mind going blank, feeling irritable, having muscle tension or sleep disturbance. This is different to the stresses and strains of everyday life.

Lyrica is a medicine that contains the active substance pregabalin. Lyrica is used to treat adults with the following conditions: GAD, neuropathic pain, or epilepsy. Lyrica is available in 25, 50, 75, 100, 150, 200, 225, and 300 mg capsules. The medicine can only be obtained with a prescription.

The recommended starting dose of Lyrica is 150 mg per day, divided into two or three doses. After three to seven days, the dose can be increased to 300 mg per day. Doses can be increased up to twice more until the most effective dose is reached. The maximum dose is 600 mg per day. Stopping treatment with Lyrica should also be done gradually, over at least a week.

Like all medicines, Lyrica can have side effects, although not everyone gets them.
Very common side-effects which may affect more than 1 person in 10 are: dizziness, tiredness.
Common side-effects which may affect more than 1 person in 100 are among others: dry mouth, nausea

Study Objectives

There were four study objectives (see table 13).

TABLE 13
Study objectives

| Study objective | Measured variable(s) | |
|---|---|---|
| 1. Evaluate the possibility to improve | Symptoms: Anxiety daytime, | Side effects: |
| clinical effect of the combination | Anxiety evening/night, Fatigue, | Dizziness, Nausea, |
| product versus PP. | Muscle tension daytime, and | and Dry mouth |
| | Muscle tension evening/night | |
| 2. Evaluate the possibility to improve | Symptoms: Anxiety daytime, | Side effects: |
| patient safety of the combination | Anxiety evening/night, Fatigue, | Dizziness, Nausea |
| product versus PP. | Muscle tension daytime, and | and Dry mouth |
| | Muscle tension evening/night | |

| | |
|---|---|
| 3. Evaluate the possibility to improve | Primary variable: Perceived Quality of Life |
| the patients' perceived Quality of | |
| Life when using the combination | |
| product. | |
| 4. Evaluate the adherence to the PP. | Primary variable: The reported intake of the PP |

Study Design and Set-Up

Three patients participated in the study, where two of them were given the combination product, A+B, and the third was given the PP, A. All three patients were using an evaluation tool in order to capture the continuous data concerning their current health situation. Prior to entering the study none of the patients had used either the PP or the CPP.

Length of study: 2 months.

Number of patients: 3

Inclusion criteria: Diagnosed GAD and access to a cellular phone capable of handling the used CPP.

Used PP: Lyrica

The two patients using the combination product were evaluated against the one patient using only the PP and being treated according to ordinary health care in Sweden. In the ordinary health care the benefits from the combination product was not possible to utilize because it was not implemented there.

The study consisted of three study set-ups (see table 14).

TABLE 14
Study set-ups GAD

| Study set- | | |
|---|---|---|
| up | Evaluation | Patients |
| 1 | A + B in detailed patient outcome | A + B: Patient 1 vs |
| | comparison with A | A: Patient 3 |
| 2 | A + B in detailed patient outcome | A + B: Patient 2 vs |
| | comparison with A | A: Patient 3 |
| 3 | A + B in comparison to | A + B: Patient 1 and 2 vs |
| | other study results | Results from published |
| | | scientific studies |

Due to the specific and profound study objectives, detailed outcome and behavior during the study period from the patients were evaluated against each other. The measurements of the patient outcomes were evaluated for each patient in different time phases during the treatment period.

In order to measure the appropriate patient outcome, the patients were continuously followed-up. In this sense, patient 3 also had a similar set of questions, which the patient answered to. The used QFM for the patients in the intervention group, patient 1 and 2:
1. Set of questions.
2. Set of functions generating individual and patient specific feedback information.
3. Feedback based upon the individual patient specific feedback information.

The used set of questions for the patient in the control group, patient 3:
1. Set of questions similar to the intervention group in order to capture patient reported outcome data
2. No set of functions.
3. No feedback information or feedback.

The used set of questions can be seen in table 15. The different questions were grouped together in questions groups with corresponding response times (see table 16) creating the question schedule. Some of the questions were asked daily, some weekly, and some were “spontaneous”, i.e., always available for the patient to answer.

TABLE 15
Questions

| Question | Question type and answer alternatives |
|---|---|
| “Are you anxious right now?” | VAS 0-10 |
| “Have you been anxious today?” | 0 = Not at all, 10 = Extremely anxious |
| “Have you been anxious during | |
| evening/night?” | |
| “Have you been tired today?” | VAS 0-10 |
| | 0 = Not at all, 10 = Extremely tired |
| “Have you felt muscle tension today?” | VAS 0-10 |
| “Have you felt muscle tension during | 0 = Not at all, 10 = Extreme muscle tension |
| evening/night?” | |
| “Have you felt any dizziness today?” | Multiple choice: Yes/No |
| “Have you felt any dizziness during | |
| evening/night?” | |
| “Have you felt any nausea today?” | Multiple choice: Yes/No |
| “Have you felt any nausea during | |
| evening/night?” | |
| “Have you felt any mouth dryness today?” | Multiple choice: Yes/No |
| “Have you felt any mouth dryness during | |
| evening/night”?” | |
| “How is your health related Quality of Life?” | VAS 0-10 |
| | 0 = Extremely bad Quality of Life, |
| | 10 = Extremely good Quality of Life |
| “Have you been able to perform everyday | Multiple choice: Yes/No |
| activities today?” | |
| “Have you exercised today?” | VAS 0-10 |
| “Have you exercised this week?” | 0 = Not at all, 10 = Exercised extremely |
| “When did you take your morning dose of | Multiple choice: Before 7 am, 7-8 am, 8-9 |
| PP?” | am, After 9 am, No morning dose |
| “When did you take your evening dose of PP | Multiple choice: Before 6 pm, 6-7 pm, 7-8 |
| yesterday?” | pm, After 8 pm, No evening dose |
| “What dose of PP did you take this morning?” | Multiple choice: 75 mg or lower, 100-175 mg, |
| | 200-275 mg, 300 mg, No morning |
| | dose |
| “What dose of PP did you take yesterday | Multiple choice: 75 mg or lower, 100-175 mg, |
| evening?” | 200-275 mg, 300 mg, No evening dose |
| “What is your current daily dose of PP?” | Numeric |
| “What is your current weight?” | Numeric |

TABLE 16
Question schedule

| Question group | Response time (alerts from CPP) |
|---|---|
| “Daily morning questions” | Daily at 10 am |
| “Daily evening questions” | Daily at 8 pm |
| “Weekly questions” | Once a week on Sundays at 8 pm |
| “Spontaneous questions” | Questions always available to answer |

Type of Feedback

The type of feedback was, as stated earlier, received personal and patient specific SMS, and feedback from the health care personnel via oral communication.

The healthcare personnel had access to updated graphs with the patient's specific feedback information based on the collected answers and the set of functions. The graphs were constructed in a way where relevant variables concerning the PP were matched together and plotted over time, examples of the matched variables are shown in table 17. An illustrative example with one of the patient's feedback graphs is shown in graph 3. Examples of given feedbacks to patients 1 and 2 were the following text messages (SMS) sent via the CPP (table 18).

TABLE 17
Example of grouping of variables in feedback graphs

| Grouping or | |
|---|---|
| feedback graphs | Question/Variable |
| Clinical effect and | “Have you been anxious today?” |
| adherence Lyrica | “Have you been tired today?” |
| | “What is your current daily dose of PP?” |
| | “What dose of PP did you take yesterday evening?” |
| | “What dose of PP did you take this morning?” |
| Side effects | “Have you felt any dizziness today?” |
| daytime and | “Have you felt any mouth dryness today?” |
| adherence Lyrica | “Have you felt any nausea today?” |
| | “What is your current daily dose of PP?” |
| | “What dose of PP did you take yesterday evening?” |
| | “What dose of PP did you take this morning?” |
| Side effects | “Have you felt any dizziness during evening/night?” |
| evening/night and | “Have you felt any mouth dryness during |
| adherence Lyrica | evening/night”?” |
| | “Have you felt any nausea during evening/night?” |
| | “What is your current daily dose of PP?” |
| | “What dose of PP did you take yesterday evening?” |
| | “What dose of PP did you take this morning?” |
| Exercise, muscle | “Have you exercised today?” |
| tension, and | “Have you felt muscle tension today?” |
| fatigue | “Have you been tired today?” |

TABLE 18
Illustrative examples of feedback messages

| | Number of SMS- | Examples of SMS message |
|---|---|---|
| Patient | messages | generated from the CPP |
| Patient 1 | 4 automatically | “Don't forget to answer the |
| | generated from the CPP | questions continuously. |
| Patient 2 | 2 automatically | If you haven't received alerts |
| | generated from the CPP | from your mobile phone recently, |
| | | please try to restart the |
| | | application again.” |

Measured Variables

The measured variables (see table 19) were symptom and side effect levels which were perceived estimates by each patient at every measure point. The change in levels of symptoms and side effects between different measured times were used for comparisons. Symptoms and side effects are many times closely related in GAD. In this study anxiety, fatigue, and muscle tension were defined as symptoms while dizziness, nausea, and dry mouth were considered as side effects. This study focused on the overall clinical effect and patient safety aspects therefore it was not crucial to the results if a measured variable could have been defined differently.

TABLE 19
Measured variables

| Symptom/Variable | Question in QFM | Question type |
|---|---|---|
| Anxiety | “Have you been anxious today?” | VAS 0-10 |
| | “Have you been anxious during | 0 = Not at all, 10 = Extremely |
| | evening/night?” | anxious |
| Fatigue | “Have you been tired today?” | VAS 0-10 |
| | | 0 = Not at all, 10 = Extremely |
| | | tired |
| Muscle tension | “Have you felt muscle tension today?” | VAS 0-10 |
| | “Have you felt muscle tension during | 0 = Not at all, 10 = Extreme |
| | evening/night?” | muscle tension |
| Dizziness | “Have you felt any dizziness today?” | Multiple choice: Yes/No |
| | “Have you felt any dizziness during | |
| | evening/night?” | |
| Nausea | “Have you felt any nausea today?” | Multiple choice: Yes/No |
| | “Have you felt any nausea during | |
| | evening/night?” | |
| Dry mouth | “Have you felt any mouth dryness | Multiple choice: Yes/No |
| | today?” | |
| | “Have you felt any mouth dryness | |
| | during evening/night”?” | |
| Health related Quality | “How is your health related quality of | VAS 0-10 |
| of Life | life?” | 0 = Extremely bad quality of |
| | | life, 10 = Extremely good |
| | | quality of life |
| Measured variable | Definition | Data type |
| Anxiety daytime | Average for variable during the period | 0-10 |
| (average) | | 0 = Not at all, 10 = Extremely |
| | | anxious |
| Anxiety evening/night | Average for variable during the period | 0-10 |
| (average) | | 0 = Not at all, 10 = Extremely |
| | | anxious |
| Fatigue (average) | Average for variable during the period | 0-10 |
| | | 0 = Not at all, 10 = Extremely |
| | | tired |
| Muscle tension | Average for variable during the period | 0-10 |
| daytime (average) | | 0 = Not at all, 10 = Extreme |
| | | muscle tension |
| Muscle tension | Average for variable during the period | 0-10 |
| evening/night | | 0 = Not at all, 10 = Extreme |
| (average) | | muscle tension |
| Dizziness (frequency) | Frequency of Yes answers for the two | 0-100% |
| | questions about dizziness during the | |
| | period | |
| Nausea (frequency) | Frequency of Yes answers for the two | 0-100% |
| | questions about nausea during the | |
| | period | |
| Dry mouth | Frequency of Yes answers for the two | 0-100% |
| (frequency) | questions about mouth dryness during | |
| | the period | |
| Health related Quality | Average for variable during the period | 0-10 |
| of Life (average) | | 0 = Extremely bad quality of |
| | | life, 10 = Extremely good |
| | | quality of life |

Study Results

The study results, measured variables and changes, are presented in the tables 20-22 below. Decimal rounding has been made to the data in the table. p.p.=percentage points. N/A=not applicable.

TABLE 20
Study results patient 1

| | | | | | | | Muscle | | | | |
|---|---|---|---|---|---|---|---|---|---|---|---|
| | | | | Anxiety | | Muscle | tension | | | | Quality |
| Phase/ | | Measured | Anxiety | Evening/ | | tension | Evening/ | | | Dry | of |
| Period | Type | variable | Daytime | Night | Fatigue | Daytime | Night | Dizziness | Nausea | mouth | Life |
| 1^{st} | A + B | Average | 4.1 | 3.6 | 5.2 | 0.9 | 0.6 | 7% | 14% | 98% | N/A |
| phase | | value | | | | | | | | | |
| | | phase | | | | | | | | | |
| 2^{nd} | A + B | Average | 4.8 | 3.8 | 2.0 | 0.0 | 0.0 | 0% | 11% | 100% | N/A |
| phase | | value | | | | | | | | | |
| | | phase | | | | | | | | | |

| | | | | | | | | | | | | |
|---|---|---|---|---|---|---|---|---|---|---|---|---|
| Change | 0.6 | 0.2 | −3.2 | −0.9 | −0.6 | −7 | p.p. | −3 | p.p. | 2 | p.p. | N/A |
| 2^{nd} vs 1^{st} | | | | | | | | | | | | |
| phase | | | | | | | | | | | | |

| | | | | | | | | | | | |
|---|---|---|---|---|---|---|---|---|---|---|---|
| 3^{rd} | A + B | Average | 3.0 | 1.3 | 1.6 | 0.0 | 0.0 | 0% | 0% | 47% | N/A |
| phase | | value | | | | | | | | | |
| | | phase | | | | | | | | | |

| | | | | | | | | | | | | |
|---|---|---|---|---|---|---|---|---|---|---|---|---|
| Change | −1.8 | −2.5 | −0.4 | 0.0 | 0.0 | 0 | p.p. | −11 | p.p. | −53 | p.p. | N/A |
| 3^{rd} vs 2^{nd} | | | | | | | | | | | | |
| phase | | | | | | | | | | | | |

| | | | | | | | | | | | |
|---|---|---|---|---|---|---|---|---|---|---|---|
| Start | A + B | Average | 4.8 | 3.8 | 5.7 | 2.1 | 1.1 | 7% | 14% | 98% | 1.0 |
| period | | value | | | | | | | | | |
| | | period | | | | | | | | | |
| End | A + B | Average | 3.2 | 1.3 | 1.7 | 0.0 | 0.0 | 0% | 0% | 47% | 2.8 |
| period | | value | | | | | | | | | |
| | | period | | | | | | | | | |

| | | | | | | | | | | | | |
|---|---|---|---|---|---|---|---|---|---|---|---|---|
| Change | −1.6 | −2.5 | −4.0 | −2.1 | −1.1 | −7 | p.p. | −14 | p.p. | −51 | p.p. | 1.8 |
| End vs | | | | | | | | | | | | |
| Start | | | | | | | | | | | | |
| period | | | | | | | | | | | | |

TABLE 21
Study results patient 2

| | | | | | | | | Muscle | | | | |
|---|---|---|---|---|---|---|---|---|---|---|---|---|
| | | | | | Anxiety | | Muscle | tension | | | | Quality |
| Phase/ | | | Measured | Anxiety | Evening/ | | tension | Evening/ | | | Dry | of |
| Period | | Type | variable | Daytime | Night | Fatigue | Daytime | Night | Dizziness | Nausea | Mouth | Life |
| 1^{st} | | A + B | Average | 4.6 | 2.3 | 4.4 | 6.0 | 4.7 | 8% | 22% | 69% | N/A |
| phase | | | value | | | | | | | | | |
| | | | phase | | | | | | | | | |
| | Start | A + B | Average | 4.8 | 4.4 | 5.4 | 7.4 | 8.0 | 13% | 33% | 0% | 5.0 |
| | period | | value | | | | | | | | | |
| | | | period | | | | | | | | | |
| | End | A + B | Average | 7.0 | 3.2 | 7.8 | 3.6 | 3.6 | 13% | 40% | 100% | 5.0 |
| | period | | value | | | | | | | | | |
| | | | period | | | | | | | | | |

| | | | | | | | | | | | | |
|---|---|---|---|---|---|---|---|---|---|---|---|---|
| Change | 2.2 | −1.2 | 2.4 | −3.8 | −4.4 | 0 | p.p. | 7 | p.p. | 100 | p.p. | N/A |
| End vs | | | | | | | | | | | | |
| Start | | | | | | | | | | | | |
| period 1^{st} | | | | | | | | | | | | |
| phase | | | | | | | | | | | | |

| | | | | | | | | | | | |
|---|---|---|---|---|---|---|---|---|---|---|---|
| 2^{nd} | B | Average | 4.2 | 2.6 | 2.7 | 4.3 | 3.8 | 0% | 14% | 0% | N/A |
| phase | | value | | | | | | | | | |
| | | phase | | | | | | | | | |

| | | | | | | | | | | | | |
|---|---|---|---|---|---|---|---|---|---|---|---|---|
| Change | −0.4 | 0.3 | −1.7 | −1.7 | −0.9 | −8 | p.p. | −9 | p.p. | −69 | p.p. | N/A |
| 2^{nd} phase | | | | | | | | | | | | |
| vs 1^{st} | | | | | | | | | | | | |
| phase | | | | | | | | | | | | |

TABLE 22
Study results patient 3

| | | | | | | | Muscle | | | | |
|---|---|---|---|---|---|---|---|---|---|---|---|
| | | | | Anxiety | | Muscle | tension | | | | Quality |
| Phase/ | | Measured | Anxiety | Evening/ | | tension | Evening/ | | | Dry | of |
| Period | Type | variable | Daytime | Night | Fatigue | Daytime | Night | Dizziness | Nausea | month | Life |
| 1^{st} | A | Average | 3.5 | 2.8 | 5.4 | 6.3 | 3.9 | 13% | 38% | 0% | N/A |
| phase | | value | | | | | | | | | |
| | | phase | | | | | | | | | |
| 2^{nd} | A | Average | 3.0 | 2.9 | 5.0 | 7.8 | 7.4 | 0% | 11% | 0% | N/A |
| phase | | value | | | | | | | | | |
| | | phase | | | | | | | | | |

| | | | | | | | | | | |
|---|---|---|---|---|---|---|---|---|---|---|
| Change | −0.5 | 0.1 | −0.4 | 1.5 | 5.5 | −13 | p.p. | −27 p.p. | 0 p.p. | N/A |
| 2^{rd} vs 1^{st} | | | | | | | | | | |
| phase | | | | | | | | | | |

| | | | | | | | | | | | |
|---|---|---|---|---|---|---|---|---|---|---|---|
| 3^{rd} | A | Average | 1.9 | 1.8 | 5.1 | 7.1 | 7.2 | 0% | 0% | 0% | N/A |
| phase | | value | | | | | | | | | |
| | | phase | | | | | | | | | |

| | | | | | | | | | | |
|---|---|---|---|---|---|---|---|---|---|---|
| Change | −1.1 | −1.1 | 0.1 | −0.7 | −0.3 | 0 | p.p. | −11 p.p. | 0 p.p. | N/A |
| 3^{rd} vs 2^{nd} | | | | | | | | | | |
| phase | | | | | | | | | | |

| | | | | | | | | | | | |
|---|---|---|---|---|---|---|---|---|---|---|---|
| Start | A | Average | 3.8 | 2.4 | 5.6 | 5.9 | 3.4 | 13% | 38% | 0% | 2.0 |
| period | | value | | | | | | | | | |
| | | period | | | | | | | | | |
| End | A | Average | 2.0 | 2.5 | 4.8 | 7.3 | 7.4 | 0% | 0% | 0% | 2.5 |
| period | | value | | | | | | | | | |
| | | period | | | | | | | | | |

| | | | | | | | | | | |
|---|---|---|---|---|---|---|---|---|---|---|
| Change | −1.8 | 0.1 | −0.8 | 1.4 | 4.0 | −13 | p.p. | −38 p.p. | 0 p.p. | 0.5 |
| End vs | | | | | | | | | | |
| Start | | | | | | | | | | |
| period | | | | | | | | | | |

The result shows a significant improvement in the clinical effect of the combination product, A+B compared to A. The level of clinical effect, concerning the measured symptoms was substantially improved for patient 1 in comparison to patient 3.

The result also shows an improvement in patient safety concerning patient 2, when the decision was taken to interrupt the treatment of the PP based on the feedback information from the combination product.

Aspects Concerning Study Set-Up 1

The results clearly show the clinical effect of the combination product versus only the PP. Patient 1 using A+B, improved in all five symptoms while patient 3, using only A, improved in just two symptoms when comparing the change in perceived symptoms from the start period to the end period. A comparison of the change, direction and magnitude, for each symptom between patient 1 and 3 shows a significant better result for patient 1 compared with patient 3, where patient 1 had better improvement in four of the five symptoms. Patient 1 also improved the perceived level of Quality of Life, meanwhile Patient 3 just improved slightly.

Some aspects of the result:
The set of functions in the QFM was crucial in order to take adequate health care decisions based upon the answers from the patients to the set of questions.
Feedback, which was a central part of the QFM, to the patient was necessary in order to gain clinical effect. When the patient got feedback on his/her answers to the set of questions, the patient's dosage of the PP was changed, which led to an increased clinical effect. The amount of side effects decreased as well.
The possibility to improve the efficacy in individualizing the dosing administration of the used PP increased substantially using the combination product, A+B. To achieve that, a QFM developed for the specific PP and the conditions of the patient, was crucial.

Review of Patient Outcomes in Set-Up 1

A central aspect with the actual PP is the titration in order to find the optimal dose for a specific patient and in this study there are several different dosing levels. In ordinary health care titration is very seldom realized. The invention, i.e., the combination product enables a new and efficient way of individualizing the dose for the specific conditions of the patient, which was seen in this study. This will be clear in the following detailed review.

Patient 1: A detailed evaluation of patient 1 for the treatment period better shows how the invention works.

During the first phase patient 1 showed symptoms of GAD and relatively low levels of side effects, with exception of dry mouth. On the basis of the answers from patient 1, feedback information generated from the set of functions indicated that a change in dosage for the actual patient would have been positive. This feedback was communicated back to patient 1.

During the second phase, now with a higher dosage of the PP, there was no improvement in two of the symptoms, but a sharp decline in the others. The side effects remained on a stable level, with a decrease in dizziness. On the basis of this information, the set of functions indicated another increase in dosage of the PP, this feedback was then communicated to patient 1.

During the third and last phase, with an even higher dosage of the PP, there was a significant improvement in two of the symptoms while the three others remained stable. The side effects decreased substantially.

The outcome of the treatment with the combination product, A+B, showed positive results comparing the start period with the end period. All five symptoms improved significantly and with no side effects with the exception of one, mouth dryness, which decreased with 50 percentage points. The perceived quality of life substantially increased compared to baseline.

Patient 3: A detailed evaluation of patient 3 for the treatment period shows a different development than for patient 1.

During the first phase patient 3 showed significant symptoms of GAD. The side effects were relatively low. There was no set of functions, no change in dosage of the PP, and no generated or communicated feedback to patient 3.

During the second phase the status of patient 3 was basically similar to the first one, but with two changes. There was deterioration in two of the symptoms and a significant decrease in the side effects. But the patient had no access to the CPP so there were no set of functions, no change in the dosage of the PP, and no feedback to patient 3.

During the third and last phase there was an improvement in two of the symptoms, but the other three remained on approximately same levels which were relatively high. The side effects were totally reset, implying a substantial decrease compared with the start period. The result of the treatment with the PP showed a mixed effect. A comparison between the start and the end period showed an improvement in two symptoms, deterioration in three symptoms, and no visible side effects at the end. The perceived Quality of Life was slightly higher in the end compared to start period.

Aspects Concerning Study Set-Up 2

The results clearly show the clinical effect of the combination product versus only the PP concerning patient safety.

Due to the fact that patient 2 is interrupted using the PP during the study period, the evaluation of patient 2 in comparison to patient 3 is of less value. This is due to the fact that the evaluation circumstances for patient 2 changes, meanwhile the circumstances for patient 3 are stable.

The evaluation of patient 2 is made on the basis that what would have been the case concerning the clinical result and patient outcome if there would have been no set of functions and no feedback to the patient. If there would have been no set of functions and no feedback to patient 2, the patient would have continued to take the PP for a period of time. Due to this, the comparison of the patient 2 development will be made between the phase after the interruption and the period before.

Some aspects of the result:
The combination product gave an improved patient safety. There was a significant improvement in both symptoms and decrease in side effects when patient 2 interrupted taking the PP. This was especially valid for three of the symptoms and all of the side effects.
The combination product, especially the QFM, was crucial in the change of using PP for patient 2. Based upon the answers to the set of questions, the feedback information generated by the set of functions indicated a change in the medication—an interruption of taking the PP. This feedback information was then communicated to the patient by the healthcare personnel
Even though patient 2 interrupted taking the PP, all five symptom values were either improved or in parity with the values during the first period. The effect of using A+B, despite the interruption of taking the PP, was positive to the patient. The corresponding situation was valid for the defined side effects.
In a comparison between patient 2 and 3, the most significant result was the dramatic change due to the interruption of taking the PP. Meanwhile patient 3 had a relatively stable development, patient 2 experienced a comprehensive change in both symptoms and side effects during the study period. A central aspect of this was the importance of the individualization enabled by the invention, and the need for a PP and patient adapted QFM permitting an individualization of the PP treatment in clinical practice.
For the complete period patient 2 improved in three symptoms and was stable in two, meanwhile patient 3 improved in one symptom, was stable in two, and deteriorated in two.

Review of Patient 2 Outcomes in Set-Up 2

During the end of the first phase the patient showed substantially deteriorations in two symptoms, improvements in two and stability in one. The side effects remained relatively high and slightly increasing. Based upon the answers from the patient, the feedback information from the set of functions indicated that the intake of the PP should be interrupted, which then was communicated to the patient through the feedback information.

During the second, and last, phase the patient was using only “B”. There was a substantial improvement in two of the symptoms and stabilization in the three others in comparison to the levels of the variables before the interruption. The levels on all measured side effects were substantially improved.

Aspects Concerning Study Set-Up 3

The results concerning non-adherence to antidepressant medication in patients with anxiety disorders, in two other published scientific studies, are reported to be 53%-70%. References: Sheehan D V, Keene M S, Eaddy M et al. CNS Drugs. 2008; 22, “Differences in medication adherence and healthcare resource utilization patterns: older versus newer antidepressant agents in patients with depression and/or anxiety disorders.” and Stein M B, Cantrell C R, Sokol M C et al, Psychiatr Serv. 2006; 57, “Antidepressant adherence and medical resource use among managed care patients with anxiety disorders.”

The average adherence to the PP concerning the patients in the intervention group was 93% which was significantly higher than the adherence to the medication for the patients in the above mentioned studies.

Even though the results from the different studies are not directly comparable due to different conditions, it is obvious that the invention in the anxiety disorders area should lead to a substantial improved adherence to a medication.

Aspects Concerning the Invention

From the results from both set-up 1 and 2, one conclusion is that the invention was central in order to achieve the results. The QFM had to be developed specifically to adapt both to the specific PP and to the patient's conditions and circumstances.

From set-up 1 it was clear that using the communication tool and the adapted QFM it was possible to improve the clinical effects of the specific PP. The specific clinical aspects of the PP were taken into consideration in the QFM and based upon the collected data and the generated feedback information, it was possible to take a decision to change the dosage of the PP. This decision was then communicated to the patient and the titration led to a positive clinical result.

In a similar way, the QFM had to be adapted to the conditions of the patient.

The results from set-up 2 illustrated in a similar way the positive effect of using the invention, including an adapted QFM, to both the specific PP and to the conditions of the patient. The decision to interrupt the medication of the actual PP was based upon the information collected through the QFM. The decision was then communicated to the patient and the result led to improved patient safety. Without the invention and an adapted QFM, it would not have been possible to get these results.

The invention makes the continuous follow-up of side effects and adverse events possible in clinical practice. The invention realizes an efficient way to detect and react on the emergence and development of side effects and adverse events.

Study 4: Ticagrelor and Acute Coronary Syndromes

Background

In this example the treatment is a combination product consisting of Brilique, the prescribed Pharmaceutical Product (PP), integrated with an interactive patient communication tool, i.e. the Computer Program Product (CPP), in accordance with the invention.

A study will be performed as described below to substantiate the efficacy of the treatment and invention. The study shall comply with national and international rules, legislation and practices with regards to e.g., ethics, informed consent, protection of confidential personal information, reporting of side effects and adverse events.

Brilique is a medicine that contains the active substance ticagrelor. It is available as round, yellow tablets (90 mg). Brilique is used together with aspirin to prevent atherothrombotic events (problems caused by blood clots and hardening of the arteries) such as heart attacks or strokes. It is used in adults who have had a heart attack or have unstable angina (a type of chest pain caused by problems with the blood flow to the heart). The medicine can only be obtained with a prescription.

Actual study population is patients with Acute Coronary Syndromes (ACS) or suffering from a myocardial infarction.

Study Objectives

The study objectives are to improve the clinical effect of the used PP, improve patient safety, and increase the patients' perceived quality of life.

The study objectives are evaluated based on the following measurements:
Prevent atherothrombotic events; increase the time to next myocardial infarction
   Heart attack
   Stroke
Control and decrease the amount of adverse events and side effects. This includes minimizing the following side effects:
   Dyspnoea (difficulty breathing)
   Epistaxis (nosebleeds)
   Gastrointestinal haemorrhage (bleeding in the stomach or gut)
   Bleeding in the skin or below the skin
   Bruising
   Bleeding at the procedural site (where a blood vessel has been punctured)
Improve the perceived Quality of Life and wellbeing.

The secondary aims concerning the studied treatment are:
Improve adherence to the medical treatment with the PP
Support lifestyle changes that have positive impact on the chosen medical treatment
   Smoke cessation (for smoking patients)
   Improved diet
   Improved physical activity

Study Design and Set Up

The study project is an open randomized study. The project comprises a total of 20 patients that will be offered access to the combination product with a specific question-feedback model (QFM). A control group is randomly chosen to be followed and receive only standard treatment and standard support in clinical practice according to ordinary health care. The patients will continuously during the study period receive questions and information through their mobile phones and computers in accordance with the set of questions specified below. The CPP and the QFM will be set up and developed in accordance with the descriptions in the documentation of the already performed studies. The specific QFM in this study will of course differ from the ones used in those studies because of the specific conditions of the chosen PP, the therapy area, and the patient group, etc.

Actual PP: Brilique, co-administered with acetylsalicylic acid (ASA).

Study length: 12 months.

Design: Open controlled study.

Population: Men and women with ACS.

Number of patients: 10 patients each in the intervention group and in the control group.

Control group: Patients taking only Brilique, co-administered with acetylsalicylic acid (ASA).

Examinations: Evaluation according to standard practice.

Sampling: Sampling according to standard practice.

Patients with ACS or suffering from a myocardial infarction are informed about the study and the possibility to enter the study. Patients willing to participate are consecutively included. Patients in the intervention group will receive a short introduction of the communication tool. If necessary, patients will be able to contact technical support for the tool. If a patient included by medical staff does not start using the tool, the tool will automatically contact the patient to offer technical support, subject to approval of the patient.

At the beginning the patient fills out a questionnaire relating to basic facts about his/her personal situation and health situation, his/her commitment to treatment, perceived commitment from medical staff, quality of life, consent to staff to retrieve data from the patient's medical records and to participate in the study. Weight, length, waist-measure and blood pressure are measured and recorded at inclusion in the study. After 12 months the same questions are asked again and the intra-individual change is calculated. Patients are informed that they shall contact medical staff if they become acutely ill or if their condition seriously deteriorates.

Criteria for inclusion:
Undergone Myocardial Infarction or unstable angina.
Access to a mobile phone capable of handling the used CPP.

Criteria for exclusion:
Patients unable to answer the questions through a mobile phone.

Description of the Used Communication Tool and Computer Program Product

A similar communication tool and computer program product as the one used in the earlier presented and performed studies will be used. See the earlier presentation of that tool for more basic details. In some areas the communication tool used in this study will differ from the earlier used one:
Improved and more distinct feedback, both to patients and to the healthcare personnel. The feedback will contain more valuable information, be easier to access and understand, and will be accessible in a variety of formats.
Improved set of functions enabling a wider possibility to identify and react upon a number of different situations concerning clinically relevant information of the specific PP, impaired progress of the patient's health situation and improved situation implying positive feedback.

The Question-Feedback Model

The Set of Questions

The set of questions will be presented to the patients according to a predefined grouping of the questions and a question schedule. The set of questions will be developed and adapted to the specific PP, Brilique.

Patient Education and Awareness

The set of questions will also take into account that patient education and health awareness are important factors for patients taking the specific PP, Brilique, especially including areas as:
Risk factors related to lifestyle, including:
   Cigarette smoking.
   Diet and diabetes.
   Low exercise.
Recognition of symptoms.
Awareness and development of medical treatment and health situation, including adherence to Brilique and treatment.

The invention can support patient education and increased awareness by, among other things:
Visualize the patient's health evolvement and important relationships.
Remind patients about their medication schedule and to increase adherence to Brilique.
Help patients to understand and detect symptoms, side effects, and adverse events of Brilique.
Support smoke cessation.
Support diet changes.
Support improved physical activity.

Individualization of the Set of Questions

The questionnaire regimen is possible to individualize according to the following aspects:
Remove some groups of questions based upon the circumstances of the specific patient
   Smoking cessation
   Extra need for improved levels of physical activity
   Extra need for improved diet
Change the response times for the questions, i.e., when the patient will be reminded to answer the questions and the current questions will appear on the patient's mobile phone.

Within each group the questions themselves are possible to further individualize.

Starting Set of Questions and Question Schedule

The questions will be given to the patients following the questions schedule as seen in table 23. This is the starting set of questions. Depending on each patient's development over time of the Brilique treatment, it can be updated in order to incorporate the specific clinical outcome. Both an update of the general set of questions and a particular update of the specific set of questions for each patient will most probably be performed, depending on clinically relevant information for the PP.

Some questions might also have different kind of dependencies, e.g., depending on the answers the questions and question schedule might alter.

TABLE 23
Questions and question schedule (illustrative)

| Question | | | | |
|---|---|---|---|---|
| group | Compulsory | Question schedule | Examples questions | |
| General | Yes for all | Month 1-2: Every | 1. | “How do you feel?” (VAS 0-10; |
| health | patients | second day | | 0 = Extremely bad, 10 = Extremely |
| status | | Month 3-4: Every | | good) |
| | | fourth day | 2. | “Are you experiencing stress |
| | | Month 5-6: Once a | | right now?” (VAS; 0 = Not at all, |
| | | week | | 10 = Extremely much) |
| | | | 3. | “Did you feel well rested this |
| | | | | morning?” (VAS; 0 = Not at all |
| | | | | rested, 10 = Extremely well |
| | | | | rested) |
| | | | 4. | “Physical exercise today, |
| | | | | example?” (Multiple choice; Sat |
| | | | | still/Standing/Partly |
| | | | | walking/Walking a lot/Hard |
| | | | | physical working) |
| | | | 5. | “What is your current daily dose |
| | | | | of Brilique? (mg]” (Numeric) |
| | | | 6. | “Do you take Brilique according |
| | | | | to the agreed prescription?” |
| | | | | (Multiple choice; Yes/Yes, |
| | | | | partially/No) |
| | | | 7. | “Have you had any social |
| | | | | activities today?” (Multiple |
| | | | | choice; Yes/Yes, partially/No) |
| | | | 8. | “Do you smoke? (Multiple |
| | | | | choice; Yes/Sometimes/No” |
| | | | | [This question will be given |
| | | | | only twice] |
| Smoke | For patients | Month 1-2: Every | 1. | “How many cigarettes have you |
| cessation | answering Yes | second day | | smoked today?” (Numeric) |
| | or Sometimes | Month 3-4: Every | 2. | “Your cravings for smoking |
| | on question #8 | fourth day | | today?” (VAS; 0 = None, |
| | in the group | Month 5-6: Once a | | 10 = Worst possible) |
| | General health | week | 3. | “How motivated are you to be |
| | status. | | | smoke free?”” (VAS; 0 = Not |
| | | | | motivated at all, 10 = Highly |
| | | | | motivated) |
| | | | 4. | “Do you have medicine for |
| | | | | smoke cessation? (Multiple |
| | | | | choice; Yes/No) |
| | | | 5. | “Do you take the medicine for |
| | | | | smoke cessation according to the |
| | | | | agreed prescription?” (Multiple |
| | | | | choice; Yes/Yes partially/No) |
| | | | 6. | “Are you satisfied with your |
| | | | | effort to quit smoking?” (VAS |
| | | | | 0-10; 0 = Can be a lot better, |
| | | | | 10 = Done my best) |
| | | | 7. | “Symptoms of urge to smoke?” |
| | | | | (Multiple choice; |
| | | | | Irritable/Depressed/Restless/Sleep |
| | | | | Disorders/Anxiety/Hunger/Concentration |
| | | | | problems) |
| | | | 8. | “Out of breath last week?” |
| | | | | (VAS 0-10; 0 = Not at all, |
| | | | | 10 = Very much) |
| | | | 9. | “Cough from smoking the last |
| | | | | week?” (VAS 0-10; 0 = No, |
| | | | | 10 = Very much) |
| Blood | Yes for all | Week 1-2: Every | 1. | “Your systolic blood pressure |
| pressure | patients | second day | | today?” (Numeric) |
| and | | Week 3-24: Once a | 2. | “Your diastolic blood pressure |
| Bleeding | | week | | today?” (Numeric) |
| | | | 3. | “Have you felt something of the |
| | | | | following?” (Multiple choice; |
| | | | | Dyspnoea (difficulty |
| | | | | breathing)/Epistaxis |
| | | | | (nosebleeds)/Gastrointestinal |
| | | | | haemorrhage (bleeding in the |
| | | | | stomach or gut)/Bleeding in the |
| | | | | skin or below the |
| | | | | skin/Bruising/Bleeding at the |
| | | | | procedural site (where a blood |
| | | | | vessel has been punctured)) |
| General | Yes for all | Month 1-3: Once a | 1. | “How have you slept this |
| health, | patients | week | | week?” (VAS 0-10; |
| treatment | | Month 4-6: | | 0 = Extremely poor, |
| | | Biweekly | | 10 = Extremely good) |
| | | | 2. | “How much have you been |
| | | | | exercising this week, compared |
| | | | | to your maximum capability?” |
| | | | | (VAS 0-10; 0 = Nothing at all, |
| | | | | 10 = At my maximal capacity) |
| | | | 3. | “To what extent has your health |
| | | | | situation affected your activities |
| | | | | this week?” (VAS 0-10; 0 = Not |
| | | | | at all, 10 = To a very large extent) |
| | | | 4. | “Your weight this morning?” |
| | | | | (Numeric) |
| | | | 5. | “Your quality of life as regards |
| | | | | to health?” (VAS 0-10; |
| | | | | 0 = Extremely bad, 10 = Extremely |
| | | | | good) |
| | | | 6. | “To what extent has your health |
| | | | | situation affected your activities |
| | | | | this week?” (VAS 0-10; 0 = Not |
| | | | | at all, 10 = To a very large extent) |
| | | | 7. | “Your weight this morning?” |
| | | | | (Numeric) |
| Side | Yes for all | Month 1-3: Once a | 1. | “Is your pulse extremely low |
| effects | patients | week | | (less than 60 beats/second)?” |
| and | | Month 4-6: | | (Multiple choice; |
| adverse | | Biweekly | | Yes/Maybe/No) |
| events | | | 2. | “Do you feel short of breath?” |
| | | | | (VAS 0-10, 0 = Not at all, |
| | | | | 10 = Very much) |
| | | | 3. | “Have you had any bleedings in |
| | | | | the following places recently?” |
| | | | | (Multiple choice; |
| | | | | Nose/Urine/Feces/Eyes/Cough/ |
| | | | | Vagina and not |
| | | | | menstrual/Strong at |
| | | | | wounds/Other strong |
| | | | | bleeding/None) |
| | | | 4. | “Have you had any of the |
| | | | | following symptoms recently? |
| | | | | (Heart attack)” (Multiple choice; |
| | | | | Discomfort in the |
| | | | | Chest/Discomfort in the Upper |
| | | | | Body/Cold Sweats/Shortness of |
| | | | | breath/Radiating pain down the |
| | | | | left arm/Squeezing chest |
| | | | | pains/Queasiness/Nausea/Upper |
| | | | | abdominal |
| | | | | pain/Weakness/Unusual |
| | | | | fatigue/Lower chest |
| | | | | pain/Indigestion like |
| | | | | symptoms/Upper back pain) |
| | | | 5. | “Have you had any of the |
| | | | | following symptoms recently? |
| | | | | (Stroke)” (Multiple choice; |
| | | | | Sudden numbness or muscles |
| | | | | contraction of the arm or leg, |
| | | | | especially on the left |
| | | | | side/Sudden fumbling in normal |
| | | | | speaking or a feeling of tied |
| | | | | tongue/Sudden drop in vision |
| | | | | with gloomy screen/Sudden |
| | | | | feeling of severe headache with |
| | | | | no reason/Loss of balance in |
| | | | | normal walking/Loss of |
| | | | | sufficient strength to stand |
| | | | | fast/Loss of consciousness with |
| | | | | fatigue/Breathing |
| | | | | trouble/Seizures like fits or |
| | | | | spasm) |
| | | | 6. | “Have you had any of the |
| | | | | following side effects recently?” |
| | | | | (Multiple choice; |
| | | | | Headache/Dizziness/Abdominal |
| | | | | pain/Diarrhea/Nausea/Rash/Itching/ |
| | | | | Gastritis) |
| | | | 7. | “Have you had any of the |
| | | | | following side effects recently?” |
| | | | | (Multiple choice; |
| | | | | Constipation/A tingling |
| | | | | feeling/Confusion) |
| General | Yes for all | Week 1-24: Every | 1. | “Number of standard measures |
| food and | patients | second week | | of alcohol last 24 hours?” |
| diet | | | | (Numeric) |
| | | | 2. | “Portion size of breakfast |
| | | | | today?” (VAS 0-10; 0 = Very |
| | | | | small, 10 = Very large) |
| | | | 3. | “Cooked meal for lunch today?” |
| | | | | (Multiple choice; Yes/No) |
| | | | 4. | “Portion size of lunch today?” |
| | | | | (VAS 0-10; 0 = Very small, |
| | | | | 10 = Very large) |
| | | | 5. | “Portion size of dinner today?” |
| | | | | (VAS 0-10; 0 = Very small, |
| | | | | 10 = Very large) |
| | | | 6. | “Are you satisfied with your |
| | | | | diet?” (VAS 0-10; 0 = Not at all |
| | | | | satisfied, 10 = Extremely |
| | | | | satisfied) |
| | | | 7. | “Are you satisfied with your |
| | | | | treatment?” (VAS 0-10; 0 = Not |
| | | | | at all satisfied, 10 = Extremely |
| | | | | satisfied) |
| Physical | For patients | Month 1: Every | 1. | “How physically active have |
| activity | who need extra | second day | | you been today?” (VAS 0-10; |
| | support to | Month 2: Twice a | | 0 = Nothing at all, 10 = At my |
| | improve their | week | | maximal capacity) |
| | physical | Month 3-6: Once a | 2. | “To what extent, in number of |
| | activity level | week | | minutes, have you been |
| | | | | physically active today?” |
| | | | | (Numeric) |
| | | | 3. | “What activities have you |
| | | | | performed today?” (Multiple |
| | | | | choice; |
| | | | | Lying/Sitting/Standing/Walking/ |
| | | | | Walking a lot/Almost |
| | | | | running/Running/Hard physical |
| | | | | working) |
| | | | 4. | “Do you feel more interested in |
| | | | | performing physical activities |
| | | | | today than a couple of days |
| | | | | ago?” (Multiple choice; Yes |
| | | | | definitely/Yes, a bit/Either |
| | | | | or/No, not really/Not at all) |
| | | | 5. | “Are you satisfied with the |
| | | | | forms of exercise you perform?” |
| | | | | (VAS 0-10; 0 = Not at all |
| | | | | satisfied, 10 = Extremely |
| | | | | satisfied) |
| Diet | For patients | Month 1: Every | 1. | “Portion size of breakfast |
| | who need extra | second day | | today?” (VAS 0-10; 0 = Very |
| | support to | Month 2: Twice a | | small, 10 = Very large) |
| | improve their | week | 2. | “Cooked meal for lunch today?” |
| | diet and food | Month 3-6: Once a | | (Multiple choice; Yes/No) |
| | intake | week | 3. | “Portion size of lunch today?” |
| | | | | (VAS 0-10; 0 = Very small, |
| | | | | 10 = Very large) |
| | | | 4. | “Portion size of dinner today?” |
| | | | | (VAS 0-10; 0 = Very small, |
| | | | | 10 = Very large) |
| | | | 5. | “Number of standard measures |
| | | | | of alcohol last 24 hours?” |
| | | | | (Numeric) |
| | | | 6. | “Are you satisfied with your |
| | | | | diet?” (VAS 0-10; 0 = Not at all |
| | | | | satisfied, 10 = Extremely |
| | | | | satisfied) |
| Follow | Yes for all | Month 1-6: Once a | 1. | “Are you going to perform any |
| up | patients | month | | surgery in the near future?” |
| (monthly) | | | | (Multiple choice; Yes/No) |
| | | | 2. | “Have you, during the last |
| | | | | month, been taking any of the |
| | | | | following medications as well?” |
| | | | | (Multiple choice; Simvastatin or |
| | | | | Lovastatin/Rifampicin/Fenytoin, |
| | | | | Karbamazepin or |
| | | | | Fenobarbital/Dexametason/Digoxin/ |
| | | | | Cyklosporin/Kinidn or |
| | | | | Diltiazem/Verapamil or a beta- |
| | | | | blocker) |
| | | | 3. | “Have you, during the last |
| | | | | month, been taking any of the |
| | | | | following medications as well?” |
| | | | | (Multiple choice; |
| | | | | Warfarin/NSAID, e.g. Ibuprofen |
| | | | | or Naproxen/SSRI. e.g. |
| | | | | Paroxetin, Sertralin or |
| | | | | Citalopram/Ketokonazol/ |
| | | | | Klaritromycin/Nefazodon/Ritonavir |
| | | | | or |
| | | | | Atazanavir/Cisaprid/Ergotalkaloider) |
| Spontaneous | Yes for all | Always accessible | 1. | “How do you feel?” (VAS 0-10; |
| | patients | for the patients to | | 0 = Extremely bad, 10 = Extremely |
| | | answer | | good) |
| | | | 2 | . “Have you had any bigger |
| | | | | bleeding recently?” (Multiple |
| | | | | choice; Yes/Maybe/No) |
| | | | 3. | “Have you had any of the |
| | | | | following symptoms recently? |
| | | | | (Heart attack)” (Multiple |
| | | | | choice; Discomfort in the |
| | | | | Chest/Discomfort in the upper |
| | | | | body/Cold sweats/Shortness of |
| | | | | breath/Radiating pain down the |
| | | | | left arm/Squeezing chest |
| | | | | pains/Queasiness/Nausea/Upper |
| | | | | abdominal |
| | | | | pain/Weakness/Unusual |
| | | | | fatigue/Lower chest |
| | | | | pain/Indigestion like |
| | | | | symptoms/Upper back pain) |
| | | | 4. | “Have you had any of the |
| | | | | following symptoms recently? |
| | | | | (Stroke)” (Multiple choice; |
| | | | | Sudden numbness or muscles |
| | | | | contraction of the arm or leg, |
| | | | | especially on the left |
| | | | | side/Sudden fumbling in normal |
| | | | | speaking or a feeling of tied |
| | | | | tongue/Sudden drop in vision |
| | | | | with gloomy screen/Sudden |
| | | | | feeling of severe headache with |
| | | | | no reason/Loss of balance in |
| | | | | normal walking/Loss of |
| | | | | sufficient strength to stand |
| | | | | fast/Loss of consciousness with |
| | | | | fatigue/Breathing |
| | | | | trouble/Seizures like fits or |
| | | | | spasm) |
| | | | 5. | “Do you feel short of breath?” |
| | | | | (VAS 0-10, 0 = Not at all, |
| | | | | 10 = Very much) |

The Set of Functions

The set of functions will take into account some of the following aspects:
Calculations in order to visualize graphs and make them clear to the actual user.
   The patients will be able to get patient specific and understandable feedback
   The authorized and responsible healthcare personnel will be able to get patient specific information in order to make decisions regarding how to increase the clinical effect and how to improve the patient safety of the specific PP.
   The patients will be given patient specific information and suggestions on how to improve the clinical effect and how to improve patient safety.
Calculations in order to find and then visualize the most interesting correlations between different outcome variables. Calculations concerning the correlation between Brilique specific factors and variables concerning clinical effect and patient safety will be focused on.
Calculations for detecting and giving immediate response to the patient, responsible healthcare personnel, and perhaps also certain authorities, when an adverse event has occurred.
Calculations for detecting and giving immediate response to the patient and responsible healthcare personnel when a serious side effect has occurred.
Calculations for detecting and giving response to the patient and responsible healthcare personnel when the patient's health situation is evolving in a negative direction.
Calculations for detecting and giving quick response to the patient and responsible healthcare personnel when other important issues have occurred.

The Type of Feedback

The type of feedback to the patients will consist of some of the following components:
Different kind of graphs based upon the answers from the patient. The feedback to the patients will, in a structured manner, visualize the correlations between different questions/variables, e.g., the result on health status when the patient is adherent to the prescribed regimen of the PP. In the graphs, the answers from the patients will, for instance, be visualized over time. This can include grouping of several variables in common graphs. The feedback information will be accessible via the patient's mobile phone and computer.
Graphs given to or accessible for the healthcare personnel. These graphs can illustrate the correlations between central outcome variables.
The graphs can contain trend lines. These will appear when the patient has been answering questions for a certain time, e.g., some trend lines will appear after a month when enough data has been reported.
The patient's evolvement over time will be illustrated in graphs, in relation to realistic health targets for that specific patient or patient group. The health targets, developed for specific questions/variables, are based upon data from earlier clinical studies/trials performed on Brilique. The targets will be individualized for each patient, depending on the amount of available information.
The patients will be sent short text messages based upon their health evolvement and adherence to the medication. These messages will be sent as encouraging and motivating information when the patient, e.g.,:
   Has fulfilled something positive, such as been adherent to the PP and/or improved their hypertension, diabetes, or blood lipids
   Needs a “small push” in order to improve their behavior, for example to become more adherent to the PP.
In relation to each patient's evolvement and the answers, future predictions concerning his/her health status will be developed. The future predictions will visualize possible scenarios for the patient based upon how the patient will evolve in some critical issues, for example the compliance/adherence to the PP in relation to health status and level of wellbeing.
Comparisons between the evolvement of the specific patient and other patients from clinical use of the PP will be made and illustrated for the specific patients. Based on this information the patients could see the possible development of their own health status by relating to the situation and progress of other patients.
The set of functions concerning possible adverse events and side effects for the PP will be implemented. The possible adverse events and/or side effects will be visualized for the patient depending on the safety concern. Possible adverse events will be sent by messages and possible side effects will be visualized in context with the given answers, for example in different graphs or other illustrations. The possible adverse events will be sent to responsible healthcare personnel as well.

In the following three studies (one prospective and two performed), the term “Software application” should be regarded as equivalent to “computer program product” and “computer application”.

In the following three patient studies, a combination product of a pharmaceutical product integrated with a mobile software application and an adapted QFM specific to the pharmaceutical was used. Test for two of the three combination products were performed, one (the first) is prospective. In the two cases which were performed, there was a period as well when the patients were using the pharmaceutical solely without the integration of the mobile software application and the specifically adapted QFM. In one of the studies the patient, on behalf of his/her own responsibility, also tested to use only the mobile software application and the QFM without using the pharmaceutical during a short period of time.

The examples showed the necessity to adapt the set of functions given the specific capabilities of the pharmaceutical, such as different levels of adherence and adverse events; and whether it is critical or not to warn the patient for particular registrations of a variable.

Study 5: Brilique (Ticagrelor)

Introduction

Development of a combination product based on the pharmaceutical Brilique and a specifically adapted QFM with a dependent software application
a. Test objectives; improved cardiovascular symptoms such as decreased level of mortality, increased level of adherence to Brilique and an improved level of physical activity through increased knowledge of own situation and awareness of adherence to Brilique
a. Follow-up variables: Level of mortality, adherence to Brilique and amount of physical activity
b. Period of time using the combination product: None; is to be initiated. This is an example of a combination product and a possible test to prove the effect of the invention.

Set of Questions Brilique

The used set of questions for the specific QFM in the combination product based on Brilique is the following:
Adherence to Brilique;
   The patient will be asked to answer a question whether or not he/she will be adherent to Brilique; “I have taken my Brilique this morning/this afternoon”. This question will show up once a day in the software application. No questions regarding dose will be given.
Physical activity;
   The patient will be asked to continuously answer a question like the following: “I have been exercising the following number of minutes today: [number]”.
Weight/BMI;
   The patient will be asked to answer a question regarding his/her actual weight.
Blood pressure;
   The patient will be asked to measure his/her actual blood pressure, either by himself/herself at home or at a clinic. Afterwards he/she is able to register it in the software application by answering a question concerning both the systolic and diastolic pressure, and where he/she had measured it; at home or at a clinic. It is possible for the patient to change or update such already registered answers.
Blood glucose;
   The patient will be asked to register the measured blood glucose, if he/she has measured it. It is possible for the patient to change or update such already registered answers.
HbA1c;
   The patient will be asked to register the HbA1c after it has been measured at a clinic.

For the defined set of questions adherence to Brilique, physical activity and weight/BMI will be prioritized in order to gain effect for the patient. The prioritization implies that the feedback messages and also the visual feedback will be focused on these questions, resulting in higher frequency of showing them, and the visual feedback will be prominent compared to the other questions.

Set of Functions Brilique

The set of functions for adherence to Brilique, and the related type of feedback, will be defined according to the following logic:
1. At a level of more than 85% of the tablets of Brilique has been taken during the last week, where the normally ordinated amount of tablet per week is fourteen, implying that no more than two missed tablets was missed, the patient shall be given a green color of the visual feedback since he/she will be regarded as adherent. In addition to that the missed tablets must not be in a row, causing a gap, in order for the patient to be regarded as adherent.
   Feedback messages encouraging the patient to remain adherent shall be given.
2. At a level of below 85%, but above 70% of the tablets of Brilique has been taken the last week, in addition to that the maximum missed tablets in a row was two, the patient shall be given a yellow color of the visual feedback.
   Feedback messages encouraging the patient to increase the level of adherence to Brilique shall be given, but they shall not be critical.
3. At a level of less than 50% of the tablets of Brilique has been taken during the last week, or if the amount of missed tablets in a row was three or more, the patient shall be given a red color of the visual feedback.
   Feedback messages encouraging the patient to promptly increase the level of adherence to Brilique shall be given, since the situation may be critical.

The set of functions for physical activity will be based on the following structure:
1. The patient reaches his/her official objective or not
2. The patient has a negative trend on physical activity based on a period of two weeks
3. The patient has a positive trend on physical activity based on a period of two weeks
4. A mix of the first point and one of the two others

The patient will be given individual feedback messages depending on which of the above criteria he/she fulfills.

The official objective can, for the Brilique QFM, be defined as zero during a period of time since some patients are ordinated not to be physically active during the first treatment period. After a period of time in combination with fulfillment from the patient of specific thresholds, the objective will be set to the ordinary level.

For weight/BMI feedback messages will be shown every second week depending on which of the following BMI levels the patient recently has registered during the last two weeks:
1. BMI between 20 and 25
2. BMI between 25 and 30
3. BMI between 30 and 35
4. BMI above 35

If the patient will have registered either a clearly decreasing or increasing trend of the BMI, the patient will be given messages concerning the purpose of either maintaining the trend or trying to interrupt it.

When the total number of patients in the test is exceeding one hundred, a change in frequency and type of messages for adherence to Brilique is performed. For patient one hundred one up to patient two hundred, the frequency of the given adherence messages will be lower and the type of messages will be a bit friendlier.

When the total number of patients in the test is exceeding two hundred, an evaluation concerning the frequency and type of given feedback messages for adherence will be performed by the set of functions. The result of the level of adherence for the first hundred patients will be compared to the result of the second hundreds of patients. If the first hundred patients are more adherent to Brilique concerning the actual period of green status for the patients, than the others, the frequency of given adherence messages will be as the used frequency for the first hundred patient. If the second hundred patients are more adherent, frequency will be increased for the first hundred. A corresponding evaluation is done concerning the level of friendliness in the messages.

When the total number of patients in the test is exceeding three hundred, a similar evaluation concerning adherence optimization is performed but in the opposite direction—given that the first hundred patients were most adherent—i.e. the evaluated frequency for new patients will be higher, i.e. more frequent, and the level of friendliness in the messages will be lower. If the second hundred patients were the most adherent in the first place, this evaluation will instead be against an even lower frequency and more friendly messages.

Corresponding evaluations is then performed, also de-coupling the level of frequency and the level of friendliness in the messages, in order to optimize the level of adherence to Brilique among the patients using the combination product. When the number of patients is exceeding five hundred a similar evaluation is performed concerning the illustration of the visual graph for the type of feedback for adherence, where different types of illustrations are compared to each other in order to optimize the level of adherence.

When the total number of patients in the test is exceeding two hundred, the 65^{th} percentile of the registered average values of performed level of physical activity from this population, will be used as the official objective for physical activity instead of the original set-up value. For every new patient this official objective will continuously be updated in order to achieve a proper objective.

When the total number of patients in the test is exceeding five hundred, the official objective for physical activity will be structured, as well, according to separate objectives for each month, based on the performed registrations from patients in the test, starting from the initiation of using the combination product. Hence, the official objective for physical activity will most probably be different for each month for the new patients using the combination product.

When the total number of patients in the test is exceeding one thousand, the official objectives for physical activity will be structured, as well, according to separate objectives for each week, based on the performed registrations from patients in the test, starting from the initiation of using the combination product. Hence, the official objective for physical activity will most probably be different for each week for the new patients using the combination product.

Type of Feedback Brilique

The following feedback components, controlled by the set of functions, will be given to the patient:
Individual, predefined messages to be shown in the software application in the patient's mobile phone. The total amount of messages may exceed two hundred. They are all kindly designed.
A simple, illustrative individual graph per variable, showing the registrations of the patient in relation to personal and official objectives for Brilique. Different amount of information will be shown for different variables.
An image/symbol indicating the actual level for the health status of each variable, illustrated as a circle with different colors and numbers within, is to be shown for the prioritized variables.
A table showing an amount of the latest registrations is to be shown in the view of the variable. From this table some of the variable registrations are possible to update.
Reminders, which the patient will be given when he/she has forgotten to register whether he/she has been adherent to Brilique or not.
General, static information without any relation to given answers from the patient. This contains information about the disease, the symptoms and the treatment.

The feedback to the patient will be immediate in the sense that also the latest registration will be able to affect the set of functions. This set-up will be verified in a small initial test prior to the example as important for achieving clinical effect, especially for the visual type of feedback.

An example of a feedback message for a patient with a green status regarding adherence to Brilique is: “It's good that you are taking Brilique as agreed upon with your doctor. By doing so you are decreasing the risk for getting a heart attack.”

A visual graph illustrating the patient adherence to Brilique the last week will be showing a diagram with fourteen different symbols for the actual seven days, since the patient shall take Brilique twice a day. If the patient doesn't answer the question whether he/she has taken Brilique for a specific occasion, a red cross is shown. If the patient will register that he/she took Brilique, a green tick is shown instead.

An example of an individual message for physical activity when the patient has fulfilled the official objectives is: “Good job! By remaining at this level of physical activity, which you are at now, a longer period of time you will substantially decrease the risk of getting another heart disease.” The patient will be given feedback messages for physical activity with a similar frequency to the patient as for adherence to Brilique.

A visual graph showing the actual achieved amount of physical activity per week the last month, for the patient will be shown in the software application. It is illustrated through different staples in relation to the official objective of the amount of physical activity.

Depending on the actual BMI level individual feedback messages shall be shown. Focus on the information in the messages is on food intake. An example of a message to a patient with BMI above 35 is: “Proper eating habits are a central part of your treatment since you have a risk for heart disease.”

A visual graph will be shown indicating the patient's actual BMI level, and in the background of the graph different colors with green for BMI less than 25; light yellow for BMI above 25 and less than 30; darker yellow for BMI above 30 and less than 35; light red for BMI above 35.

For blood pressure, blood glucose and HbA1c only general feedback messages will be given to the patient without relation in the set of functions to the actual registered patient values. The messages will be focusing on general health, such as physical activity and food intake, but also mention blood pressure, blood glucose and HbA1c in order to make the patient aware of them. For the three variables visual graphs are to be shown for the actually registered patient values.

Study 6: Zoloft (Sertralin)

Introduction

Development and test of a combination product based on the pharmaceutical Zoloft and a specifically adapted QFM with a dependent software application
a. Test objectives; improve cardiovascular and diabetes symptoms through an increased well-being from improved adherence to Zoloft, initiating primarily an improved level of physical activity
b. Follow-up variables: Weight and HbA1c
c. Period of time using the combination product: Five months.
d. Period of time using only the pharmaceutical, i.e. no combination product, prior to the period of using the combination product: Two months
e. Period of time using only the software application, after the period of using the combination product: Two weeks

Set of Questions Zoloft

The used set of questions for the specific QFM in the combination product based on Zoloft was the following:
Adherence to Zoloft:
   The patient was asked to answer a question whether or not he/she had been adherent to Zoloft, and which dose the patient had taken; “I have taken my Zoloft today with the dose 25 mg/50 mg/100 mg/150 mg or 200 mg”.
Physical activity:
   The patient was asked initially to set up an individual goal with the purpose of achieving an increased effect. The individual goal was set-up by the patient by answering the following question: “Give your own personal goal for the physical activity in number of minutes for one week”.
   The patient was then asked to continuously answer a question like the following: “I have been exercising the following number of minutes today: [number]”.
Weight/BMI:
   The patient was asked to answer a question regarding his/her actual weight.
Depression and Anxiety, respectively:
   The patient was asked to register the actual level of perceived depression respectively actual level of perceived anxiety at predefined occasions every second day. It was also possible for the patient to answer the question when he/she wanted. The questions were structured as a Visual Analog Scale (VAS).
Stress:
   The patient was asked to register the actual level of perceived stress. The question did show up at predefined occasions every second day. It was also possible for the patient to answer the question when he/she wanted. The question was structured as a VAS.
HbA1c:
   The patient was asked to register his/her HbA1c after it had been measured at a clinic.
Three specific possible adverse events for Zoloft:
   “Do you have severe skin rash in your mouth or tongue? Extremely skin rash versus No skin rash at all” according to a Visual Analogue Scale
   “Do you experience symptoms such as itchy rash, respiratory problems, wheezing or swellings in your face? Extremely much versus Nothing at all” according to a VAS structure
   “Have you been upset or confused; or have you had diarrhea, fever and high blood pressure; or have you had excessive sweating and rapid heartbeat? Extremely much versus Nothing at all” according to a VAS structure

All of the questions were equally prioritized, in order to gain effect for the patent, except for HbA1c, Anxiety, Stress and the adverse events. The prioritization implied that the feedback messages and also the visual feedback were focused on these questions, resulting in higher frequency of showing them, and the visual feedback was prominent compared to the other questions.

Set of Functions Zoloft

The set of functions for adherence to Zoloft, and the related type of feedback, were defined according to the following logic:
1. At a level defined as a period of only one missed occasion, or less, to take tablet(s), i.e. not two missed occasions taking tablets in a row, a general type of feedback messages was shown to the patient every second day indicating he/she was adherent. The patient was also given a green color on the visual feedback.
2. At a level of two missed occasions to take tablets, or more, in a row, the patient was regarded as non-adherent. Another type of message was shown to the patient every second day. The patient was given a red color on the visual feedback.

The set of functions for physical activity was utilizing both personal and official goals. The personal goal could, for the Zoloft QFM set-up, be updated by the patient whenever he/she wanted. The physical activity official goal was higher than for the case with, for example, Brilique.

The patient was given feedback messages for physical activity using the following structure:
1. The use of individual goals or not
2. The patient reaches his/her individual goal or not
3. The patient reaches his/her official goal or not

The patient was given individual feedback messages depending on which of the above levels he/she registered.

For weight/BMI feedback messages were sent depending on which of the following BMI levels the patient recently had registered during the last two weeks:
1. BMI between 20 and 25
2. BMI between 25 and 30
3. BMI between 30 and 35
4. BMI above 35

Set of functions for both Depression and Anxiety was configured to detect a predefined amount of registrations above a certain level of the variable performed during a specific time interval; at least three registrations above the level eight during at least three days. When that criterion was fulfilled a predefined message was shown to the patient.

Set of functions for Stress and HbA1c didn't cause any feedback to the patient.

All of the three specific adverse events for Zoloft were set-up using the following logic:
1. If any of the questions resulted in a registration on the VAS exceeding level five on the ten grade scale, a message was shown to the patient that he/she should contact his/her responsible doctor and describe his/her situation
2. If any of the questions resulted in a registration on the VAS exceeding level seven on the ten grade scale, a message was shown to the patient that he/she should promptly contact his/her responsible doctor and describe his/her situation
3.

Type of Feedback Zoloft

The following feedback components, controlled by the set of functions, were given to the patients:
Individual, predefined messages shown in the software application in the patients' mobile phone. The amount of messages exceeded fifty and they were all relatively kindly designed.
A simple, illustrative individual graph per variable, showing the registrations of the patient in relation to the personal and the official objectives for Zoloft. Different amount of information was shown for different variables.
An image/symbol indicating the actual level for the health status of each prioritized variable, illustrated as a circle with different colors and numbers within, was shown for the prioritized variables.
General, static information without any relation to given answers from the patient.

The feedback to the patient was immediate in the sense that also the latest registration should be able to affect the set of functions.

An example of a feedback message for a patient with a green status on adherence to Zoloft was: “It's good that you are taking Zoloft according to your prescription. By doing so you are helping yourself”. An example of an adherence message with red status was: “Don't miss to take Zoloft. It might result in difficulties for you”.

A visual graph illustrating the patient adherence to Zoloft the last ten days showed a diagram with ten different symbols for the actual ten days, since the patient should take Zoloft once a day. If the patient didn't answer the question whether he/she has taken Zoloft for a specific occasion, a red cross was shown. If the patient had registered that he/she took Zoloft, a green tick was shown instead.

The patient was given feedback messages on performed physical activity depending on which of the above levels he/she registered. An example of a message when the patient has reached the official goal: “Good job! By being physically active you will not only feel better, but you will probably fight your disease efficiently as well.”

A visual graph showing the actual achieved amount of physical activity per week, for the last two months, was shown in the software application. It was illustrated through different staples in relation to both the personal goal and the official goal of amount of physical activity.

Depending on the actual BMI level feedback messages were shown. Focus on the information in the messages was food, but also physical activity. An example of a message sent to a patient with BMI above 35 was: “Here are three good reasons to start walking: improved ability to concentrate, improved sleep and increased immunity.”

A visual graph was shown indicating the patient's actual BMI level, and in the background of the graph different colors with green for BMI less than 25; light yellow for BMI above 25 and less than 30; darker yellow for BMI above 30 and less than 35; light red for BMI above 35.

For Depression, Anxiety and Stress a visual graph, respectively, was illustrating the patient's registrations in the software application. If the patient registered answers fulfilling the criteria for Depression or Anxiety, a feedback message like the following was shown to the patient: “You have answered relatively high values on Depression resp. Anxiety and you should contact your responsible doctor and tell him/her about your situation and how you feel.”

For both Stress and HbA1c visual graphs were shown, respectively, to illustrate the registrations.

For the possible adverse events, the following message was shown to the patient if he/she had registered on level one; “You seem to have . . . [the actual symptom] and should contact your responsible doctor and tell him/her about your situation and how you feel.” If the patient registered on level two, the following message was shown: “You seem to have . . . [the actual symptom] and should promptly contact your responsible doctor and tell him/her about your situation and how you feel, if you haven't already done it.”

Test Results Combination Product Zoloft

Baseline value before test; HbA1c: 66 mmol/mol and Weight: 102 kg

End value after test; HbA1c: 55 mmol/mol and Weight: 96 kg

During the test period of using the combination product based upon Zoloft and a specifically adapted QFM with a dependent software application, the patient decreased 11 mmol/mol in HbA1c, and 6 kg of weight, implying a decrease of 17% in HbA1c and 6% in weight.

The achieved results are not typical for Zoloft, but more an example of the combination product. Through a better well-being and motivation for an increase in physical activity, the patient both loses weight and improves the level of HbA1c. The patient also experienced that he/she was feeling better, the levels of anxiety and stress decreased, however no formal measurements were made on those variables. The actual dose of Zoloft was changed twice during the period, starting at 100 mg and ending on 25 mg.

During the test period when the patient only was taking Zoloft, i.e. the full combination product was not used, the HbA1c remained stable or slightly increased, and meanwhile the weight gained 1-2%.

During the short test period when the patient only used the software application, i.e. the patient did not use the combination product or take Zoloft, the patient quite immediately registered increased values of both Depression, and especially Anxiety. This period of test was therefore interrupted after just a short period of time, clearly indicating that the combination product also including Zoloft is superior to only using the software application in this case.

Study 7: Metformin

Introduction

Development and test of a combination product based on the pharmaceutical Metformin and a specifically adapted QFM with a dependent software application
a. Test objectives; improved diabetes symptoms through primarily an increased level of physical activity, and improved adherence to Metformin
b. Follow-up variables: HbA1c
c. Period of time using the combination product: Three months.
d. Period of time using only the pharmaceutical, i.e. no combination product, prior to the period of using the combination product: Two months

Set of Questions Metformin

The used set of questions for the specific QFM in the combination product based on

Metformin was the following:
Adherence to Metformin:
   The patient was asked to answer a question whether or not he/she had been adherent to Metformin, and which dose the patient took; “I have taken my Metformin today with the daily dose of 500 mg/1000 mg/1500 mg/2000 mg/2500 mg or 3000 mg”.
Physical activity:
   The patient was asked to continuously answer a question like the following: “I have been exercising the following number of minutes today: [number]”.
Weight/BMI:
   The patient was asked to answer a question regarding his/her actual weight.
Blood glucose:
   The patient was asked to register his/her measured blood glucose, when he/she had measured it. It was possible for the patient to change or update already registered answers.
HbA1c:
   The patient was asked to register his/her HbA1c after it had been measured at a clinic.
A possible adverse event for Metformin:
   “Have you experienced unexpected loss of weight, severe nausea or vomiting (malaise), uncontrolled sudden pain when breathing or abdominal? Extremely much versus Nothing at all” according to a VAS structure
A possible side effect of Metformin:
   “Have you experienced diarrhea, decreased appetite, malaise or abdominal pain particularly during the initial treatment? Extremely much versus Nothing at all” according to a VAS structure

All of the questions were equally prioritized, in order to gain effect for the patent, except for HbA1c and the adverse events. The prioritization implied that the feedback messages and also the visual feedback were focused on these questions, resulting in higher frequency of showing them, and the visual feedback was prominent compared to the other questions.

Set of Functions Metformin

The set of functions for adherence to Metformin, and the related type of feedback, was defined according to the following logic. One occasion was defined as a daily dose of Metformin.
1. At a level defined as a period of only one missed occasion, or less, to take tablet(s), i.e. not two missed occasions taking tablets in a row, and a maximum of totally two missed occasions a week, a general type of feedback message was shown to the patient every third day indicating he/she was adherent. The patient was also given a green color on the visual feedback.
2. At a level defined as a period of maximum of two missed occasions to take tablets in a row, or three missed occasions the last week, the patient was regarded as non-adherent but not critical. Another type of message was shown to the patient every third day. The patient was given a yellow color on the visual feedback.
3. At a level defined as a period of three missed occasions to take tablets in a row or more, or totally four or more missed occasions the last week, the patient was regarded as non-adherent and critical. Another type of message was shown to the patient every third day. The patient was given a red color on the visual feedback.

The set of functions for physical activity was utilizing the official objective for physical activity, which was higher than for both Brilique and Zoloft.

The patient was given feedback messages for physical activity using the following structure:
1. The patient reaches his/her official objective or not
2. The patient has a negative trend on physical activity based on a period of two weeks
3. The patient has a positive trend on physical activity based on a period of two weeks

The patient was given individual feedback messages depending on which of the above criteria he/she fulfilled.

For weight/BMI feedback messages were sent depending on which of the following BMI levels the patient recently had registered during the last ten days:
1. BMI between 20 and 25
2. BMI between 25 and 30
3. BMI above 30

Set of functions for Blood glucose, connected to that specific question, was configured to detect both the hyperglycemia and the hypoglycemia of the patient. A predefined amount of registrations above a defined level for hyperglycemia or below another for hypoglycemia triggered predefined messages.

If the patient registered blood glucose three times in a row exceeding 15 mmol/L messages for hyperglycemia were shown to the patient and registrations at only one occasion below 2,5 mmol/L a message for hypoglycemia was shown.

Set of functions for HbA1c didn't cause any feedback to the patient.

Set of functions for the possible adverse event was according to the following logic:
1. If the question resulted in a registration on the VAS exceeding level five on the ten grade scale, a message was shown to the patient that he/she should contact his/her responsible doctor and describe his/her situation
2. If the question resulted in a registration on the VAS exceeding level seven on the ten grade scale, a message was shown to the patient that he/she should promptly contact his/her responsible doctor and describe the situation

Set of functions for the side effect was according to the following step:
If the question resulted in a registration on the VAS exceeding level seven on the ten grade scale, a message was shown to the patient that he/she should contact his/her responsible doctor and describe the situation

Type of Feedback Metformin

The following feedback components, controlled by the set of functions, were given to the patient:
Individual, predefined messages shown in the software application in the patient's mobile phone. The amount of messages exceeded sixty and they were all kindly designed.
A simple, illustrative individual graph per variable, showing the registrations of the patient in relation to the objectives for Metformin. The time scales differed between the different variables.
An image/symbol indicating the actual level for the health status of each prioritized variable, illustrated as a circle with different colors and numbers within, was shown for the prioritized variables.
General, static information without any relation to given answers from the patient.

The feedback to the patient was immediate in the sense that also the latest registration should be able to affect the set of functions.

An example of a feedback message for a patient with a green status on adherence to Metformin was: “It's good that you are taking Metformin according to your prescription. By doing so you are improving your situation with diabetes”. An example of an adherence message with red status was: “You shouldn't miss taking Metformin, it will help you with your diabetes”

A visual graph illustrating the patient adherence to Metformin the last week showed a diagram with seven different symbols for the actual seven days, one for each occasion the patient is taking the daily dose. If the patient didn't answer the question whether he/she had taken Metformin for a day, or denied to take it, a red cross was shown. If the patient had registered that he/she had taken Metformin, a green tick was shown instead in the diagram.

The patient was given feedback messages depending on which of the criteria of physical activity he/she fulfilled. An example of a message when the patient has reached the official objective: “Really good job with your exercise! By being physically active your heart will be more powerful and your resting heart rate will decrease, and your muscles will increase”. Corresponding messages were also shown if the patient fulfilled criteria number three.

A visual graph showing the actual achieved amount of physical activity per week, for the last two months, was shown in the software application. It was illustrated through different staples in relation to the official objective of amount of physical activity.

Depending on the actual BMI level feedback messages were shown. Focus on the information in the messages was food and physical activity. An example of a message sent to a patient with BMI above 30 was: “By losing weight you will get several positive effects such as improved blood glucose control, reduced lipids and decreased blood pressure.”

A visual graph was shown indicating the patient's actual BMI level, and in the background of the graph different colors with green for BMI less than 25; light yellow for BMI above 25 and less than 30; light red for BMI above 30.

When the set of functions triggered a condition of hyperglycemia for the patient, a corresponding message was shown: “You seem to have had a little too high value on blood glucose. Hence, think of both being adherent to Metformin and having a proper intake of energy. If you have any questions, you could contact your responsible doctor”.

When the set of functions triggered a condition of hypoglycemia, a corresponding message could be shown: “You seem to have low blood sugar. If you haven't already done it, you should immediately take some sugar. If this frequently happens you should contact your responsible doctor”.

HbA1c registrations were illustrated in a graph, but didn't cause any feedback messages to the patient.

For the possible adverse event according to the set of functions, a corresponding message was shown to the patient if he/she fulfilled level one; “You seem to have problems with your diabetes and should contact your responsible doctor and tell him/her about your situation and how you feel.” If the patient fulfilled level two, the following message was shown: “You seem to have severe problems with your diabetes and should promptly contact your responsible doctor and tell him/her about your situation and how you feel.”

For the possible side effect according to the set of functions, a corresponding message was shown to the patient; “You seem to have had a side effect and should contact your responsible doctor and tell him/her about your situation and how you feel.”

Test Results Combination Product Metformin:

Baseline value before test; HbA1c: 55 mmol/mol

End value after test; HbA1c: 48 mmol/mol

During the actual period of time of using the combination product based upon Metformin and a specifically adapted QFM with a dependent software application, the patient decreased 7 mmol/mol in HbA1c implying a decrease of 13%. The actual dose of Metformin was changed twice during the period, starting at 500 mg and ending at 2000 mg.

During the test period when the patient was only taking Metformin, i.e. the full combination product was not used, the HbA1c slightly increased with 7%.

1. Substance with pharmaceutical activity against a medical condition for use in a treatment of said medical condition in combination with a computer program product comprising instructions causing a computer to perform a method comprising the steps
providing a patient with a set of questions according to a question schedule, wherein said set of questions is adapted to the pharmaceutical product;
collecting answers to said questions from said patient;
subjecting said answers to a set of functions adapted for the set of questions and the pharmaceutical product thereby generating patient-specific feedback information;
providing said feedback information to the patient; and
extracting information from said answers and providing said information to a database adapted for collecting information during clinical use of said substance, wherein said database is adapted to store information comprising one or more of: patient identifier, respondent identifier, individual caregiver identifier, organizational caregiver identifier, substance identifier, substance combination identifier, respondent answers, type and date of occurrence of adverse events, type and degree of adverse effects of one or more substance or substance combination, probability of an adverse event, probability of an adverse effect, patient health status, patient history, patient family history, patient genetic information, prescribed dosage or administration regimen, drug-drug interactions, life style factors.

2. Substance according to claim 1, wherein the computer program product comprises instructions causing a computer to perform a method comprising the steps
providing at least one further respondent in addition to said patient with a second set of questions according to a second question schedule, wherein said second set of questions is adapted to the pharmaceutical product;
collecting answers to said questions from said further respondent;
subjecting said answers from said further respondent to a second set of functions adapted for the second set of questions and the pharmaceutical product thereby generating patient-specific feedback information;
providing said feedback information to the patient and, optionally, to the further respondent; and
extracting information from said answers from said further respondent and providing said information to a database adapted for collecting information during clinical use of said substance, wherein said database is adapted to store information comprising one or more of: patient identifier, respondent identifier, individual caregiver identifier, organizational caregiver identifier, substance identifier, substance combination identifier, respondent answers, type and date of occurrence of adverse events, type and degree of adverse effects of one or more substance or substance combination, probability of an adverse event, probability of an adverse effect, patient health status, patient history, patient family history, patient genetic information, prescribed dosage or administration regimen.

3. Substance according to claim 1, wherein the computer program product comprising instructions causes a computer to perform a method comprising the steps
a) providing a patient and optionally a further respondent with sets of questions according to a question schedule, wherein said sets of questions are adapted to the pharmaceutical product;
b) collecting answers to said questions from said patient and optionally said further respondent;
c) subjecting said answers to a set of functions adapted for the sets of questions and the pharmaceutical product thereby generating patient-specific feedback information;
d) providing said feedback information to the patient and optionally to the further respondent;
e) extracting information from said answers and providing said information to said database adapted for collecting information during clinical use of said substance;
f) providing information stored in said database to a reviser subjecting the sets of questions and/or the sets of functions to a revision based on said information stored in said database;
g) obtaining a revised set of questions and/or a revised set of functions from said reviser; and
h) repeating steps a)-g).

4. Substance according to claim 1, wherein said database is adapted for storing information collected from more than one patient, preferably at least 50%, such as at least 75% or substantially 100% of patients, clinically using said combination of substances in combination with said computer program product.

5. Substance according to claim 3, wherein said revision is based on information collected from said patient and/or other patients clinically using said combination of substances in combination with said computer program product.

6. Substance according to claim 3, wherein said revision is based on information obtained during clinical trials of the substance and/or commercial use of the substance.

7. Substance according to claim 1, wherein said database is adapted to store information comprising two, five, ten, fifteen, or more of: patient identifier, respondent identifier, individual caregiver identifier, organizational caregiver identifier, substance identifier, substance combination identifier, respondent answers, type and date of occurrence of adverse events, type and degree of adverse effects of one or more substance or substance combination, probability of an adverse event, probability of an adverse effect, patient health status, patient history, patient family history, patient genetic information, prescribed dosage or administration regimen, drug-drug interactions, life style factors.

8. A substance according to claim 1, wherein the clinical relevance of the combination of said set of questions and said set of functions has been validated in clinical trials.

9. A substance according to claim 1, wherein said set of questions and said set of functions are related to patient compliance to a preferred or prescribed dosage and/or administration regimen of said pharmaceutical product.

10. A substance according to claim 1, wherein said set of questions and said set of functions are related to an indication of possible occurrence or development of an adverse event and/or side effect.

11. A substance according to claim 1, wherein said set of questions and said set of functions are related to the patient's quality of life.

12. A substance according to claim 1, wherein at least a subset of the set of questions is related to the actual administration; actual dosage; perceived and/or measured therapeutic effects; test results and/or perceived quality of life.

13. A substance according to claim 1, wherein the method further comprises subjecting said answers to a set of functions adapted for the set of questions and the pharmaceutical product thereby generating an updated question schedule, wherein said set of functions optionally use Computer Adaptive Testing and/or Item Response Theory.

14. A substance according to claim 1, wherein said set of functions include functions selected from the group consisting of: calculations of target parameters and trend lines; prediction of development of a condition; rules and thresholds for defining when to give notifications.

15. A substance according to claim 1, wherein said computer program product comprises instructions causing a computer to provide feedback selected from graphs, diagrams, graphical illustrations and text messages.

16. A substance according to claim 1, wherein said method provides feedback only to the patient, or to the patient and to other individuals.

17. A substance according to claim 1, wherein said computer program product is provided on a physical medium or by means or instructions for accessing and installing the computer program product on a computer.

18. A kit of parts comprising a substance and a computer program product according to claim 17, wherein said computer program product is provided by means or instructions for accessing and installing the computer program product on a computer and said kit further comprises an identifier unique to the kit.

19. A substance according to claim 1, wherein said substance is selected from the group consisting of Aripiprazol (Abilify), Rimonabant (Acomplia), Pioglitazon (Actos), glucoseamine (Glucosine), Octocog alfa (Adnate, Advair), Flutikason in combination with Salmeterol (Seretide), zolpidem (Ambien, Stilnox), Insulin glulisin (Apidra), Donepezil (Aricept), irbesartan (Avapro, Aprovel), rosiglitazone (Avandia), metformin in combination with rosiglitazone (Avandamet), glimepiride in combination with rosiglitazone (Avandaryl), bevacizumab (Avastin), Interferon beta (Avonex), Darbepoetin alfa (Aranesp), anastrozole (Arimidex), Kandesartan (Atacand), olmesartan (Benicar, Olmetec), Interferon beta-lb (Betaseron), Interferon beta (Betaferon), exenatide (Byetta), Bikalutamid (Casodex), Celecoxib (Celebrex, Celebra), Escitalopram (Cipralex/Lexapro), duloxetine (Cymbalta), Vareniklin (Champix), Glatiramer (Copaxone), Carvedilol (Coreg), Losartan (Cozaar), Rosuvastatin (Crestor), Ramipril (Tritace), Valsartan (Diovan), Venlafaxin (Efexor), oxaliplatin (Eloxatin), Etanercept (Enbrel), raloxifene (Evista), ezetimibe (Ezetrol, Zetia), Tamsulosin (Flomax, Flomaxtra, Urimax), fluticasone (Flovent, Flixotide), Alendronic acid (Fosamax), Gemcitabine (Gemzar), imatinib mesylate (Gleevec, Glivec), Trastuzumab (Herceptin), insulin lispro (Humalog), Adalimumab (Humira), Lopinavir/ritonavir (Kaletra), Sumatriptan (Imitrex, Imigran), Sitagliptin (Januvia), insulin glargin (Lantus), Fenofibrate (Lipanthyl, TriCor), atorvastatin (Lipitor), Insulin Detemir (Levemir), amlodipine and benazepril (Lotrel), Leuprorelin, (Lupron, Leuplin), pregabalin (Lyrica), rituximab (Mabthera, Rituxan), Telmisartan (Micardis), Esomeprazole (Nexium), amlodipine (Norvasc), insulin aspart (NovoLog, NovoMix, NovoRapid), repaglinid (NovoNorm), Rabeprazole (Pariet), paroxetine (Paxil, Seroxat), Pantoprazole (Protonix, Pantozol, Pantoloc), Clopidogrel (Plavix), pravastatin (Pravachol), Epoetin Alfa (Procrit, Eprex), takrolimus (Protopic), budesonid (Pulmicort), interferon beta-1a (Rebif), sibutramin (Reductil), Infliximab (Remicade), Risperidon (Risperdal), Metoprolol (Seloken, Toprol), quetiapine (Seroquel), Tiotropium (Spiriva), budesonide and formoterol (Symbicort), Montelukast (Singulair), Docetaxel (Taxotere), Topiramat (Topamax), Emtricitabin and Tenofovirdisoproxil (Truvada), ezetimibe and simvastatin (Vytorin), bupropion (Wellbutrin), Betametason in combination with Kalcipotriol (Xamiol) calcipotriene (Taclonex), simvastatin (Zocor), Sertralin (Zoloft), zoledronic acid (Zometa), Olanzapin (Zyprexa), cetirizine (Zyrtec), ticagrelor (Brilique).

20. The computer program product of claim 1.

21. The method of claim 1.
